(19) United States
(12) Patent Application Publication
(10) Pub. No.: US 2008/0269161 A1
(43) Pub. Date: Oct. 30, 2008

(54) Compositions and Methods Relating to Mitochondrial Hyperpolarization in Neurological Disease

(76) Inventors: Seth Perry, Rochester, NY (US); John Phillip Norman, New York, NY (US); Stephen Dewhurst, Rochester, NY (US); Harris A. Gelbard, Pittsford, NY (US)

Correspondence Address: Ballard Spahr Andrews & Ingersoll, LLP, SUITE 1000, 999 PEACHTREE STREET, ATLANTA, GA, 30309-3915, US

(21) Appl. No.: 11/795,664

(22) PCT Filed: Jan. 19, 2006
(86) PCT No.: PCT/US06/01987

§ 371 Date: May 30, 2008

Related U.S. Application Data
(60) Provisional application No. 60/645,426, filed on Jan. 20, 2005.
(60) Provisional application No. 60/663,424, filed on Mar. 18, 2005.

(51) Int. Cl. A61K 31/708; C12N 5/02; A61K 31/551; A61K 31/4725; A61K 31/427; A61K 31/472; A61K 31/536; A61K 31/444; A61K 31/496; A61K 31/341; A61K 31/513; A61K 31/4402; A61K 31/7072

(52) U.S. Cl. 514/45; 435/375; 435/29; 514/220; 514/307; 514/365; 514/253.09; 514/471; 514/274; 514/357; 514/50; 514/230.5

(57) ABSTRACT

Provided is a method of protecting a neuron from dysfunction induced by an HIV neurotoxin comprising contacting the cell with a therapeutically effective dose of an inhibitor of mitochondrial hyperpolarization.

### CROSS-REFERENCE TO RELATED APPLICATIONS

This application claims benefit of U.S. Provisional Application No. 60/645426, filed Jan. 20, 2005 and of U.S. Provisional Application No. 60/663424, filed Mar. 18, 2005, which are hereby incorporated herein by reference in their entirety.

### ACKNOWLEDGEMENTS

This work was supported by: National Institute of Mental Health P01MH64570; National Institute of Mental Health MH56838; NIEHS Training Grant ES07026 (J. P. Norman) and National Institute of Allergy and Immunology T32 AI49815. The Government has certain rights in the invention.

### BACKGROUND OF THE INVENTION

Despite the efficacy of highly active antiretroviral therapy (HAART) in reducing viral burden, neurologic disease associated with HIV-1 infection of the central nervous system CNS has not decreased in prevalence. HIV-1 does not induce disease by direct infection of neurons, although extensive data suggest that intra-CNS viral burden correlates with both the severity of virally-induced neurologic disease, and with the generation of neurotoxic metabolites. Many of these molecules are capable of inducing neuronal apoptosis in vitro, but neuronal apoptosis in vivo does not correlate with CNS dysfunction. Thus, the mechanism of virally-induced neurologic disease is not known in the literature.

### BRIEF SUMMARY OF THE INVENTION

Provided is a method of protecting a neuron from dysfunction induced by an HIV neurotoxin comprising contacting the cell with a therapeutically effective dose of an inhibitor of mitochondrial hyperpolarization.

Also provided is a method of treating or preventing HIV-1 associated dementia (HAD) in a subject in need of such treatment or prevention, comprising administering to the subject a therapeutically effective dose of an inhibitor of mitochondrial hyperpolarization.

Further provided is a method of identifying a compound that can promote neural cell protection, the method comprising contacting a neural cell with a candidate neural protecting compound, contacting the neural cell with an agent that induces mitochondrial hyperpolarization, and evaluating the ability of the compound to prevent or inhibit mitochondrial hyperpolarization in the cell.

Also provided is a composition, comprising a molecule that inhibits mitochondrial hyperpolarization in a neural cell and an antiretroviral compound.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows that Tat and PAF increase synaptic vesicular activity. Images of (A) Brightfield and (B) fluorescent FM1-43 labeling show that FM1-43 uptake results in labeling that is punctate and oriented primarily along neuronal processes. (C) Moreover, activity dependent FM1-43 release by KC1 depolarization preferentially reduces the punctate vesicular labeling versus background signal. (D) In ≦14 and ≧14 day old primary neurons, 2.5 μg/ml Tat and 4.25 μg/ml Tat induces an increase in spontaneous activity dependent vesicular uptake, an effect that is both dose and culture age dependent. (E) However, no effect is seen with mutated Tat protein, suggesting that Tat's effect is due to biologically specific activities of its functional region. (F) 464 nM PAF had an even greater effect on FM1-43 uptake at 24 hours.

FIG. 2 shows that Tat and PAF increase ROS in cortical neuronal cultures. Treatment of rat cortical neurons for 24 hours with Tat or PAF induced elevated levels of ROS versus control, as measured with oxidizable dye indicator 5-(and-6)-chloromethyl-2′,7′dichlorodihydrofluorescein diacetate, acetyl ester (CM-H2DCFDA) (DCF). This effect was greatest with 2.5 μg/ml Tat, resulting in a 110% increase in ROS production (*p<0.0001 versus control), whereas 464 nM PAF resulted in a 50% increase in ROS production, and the H202 positive control induced a 90% increase.

FIG. 3 shows that antioxidants ameliorate the Tat-induced rise in vesicular uptake of neurotransmitter. (A) The antioxidant TUDCA completely eliminated Tat's effects on FM1-43 uptake for all doses of Tat.

FIG. 4 shows that Tat and PAF cause a dose-dependent mitochondrial hyperpolarization in cortical neurons. (A) Treatment of rat cortical neurons with 100 ng/ml or 2.5 μg/ml Tat for 1, 4, 10, 24, 26, 36, or 48 hours resulted in a dose-dependent biphasic increase in mitochondrial membrane potential. At each dose, initial peaks were followed by periods of apparent mitochondrial membrane potential (ΔΨₘ ) stabilization, followed by increased ΔΨₘ again at later time points, and the later increase in ΔΨₘ persisted until the end of the analysis (48 hours). A biologically inactive mutated Tat peptide (green) produced no effect. Curve fits are non-formulaic software interpolations of the data points. (B) Like Tat, its downstream mediator PAF also induced a dose-dependent rise in ΔΨₘ (*p <0.002 or p <0.0001 versus control, respectively) C) Effect of high dose FCCP (5 μM) on mitochondrial TMRM uptake at 1 hour is shown. This loss of TMRM signal with 5 μM FCCP (which would induce strong mitochondrial depolarization) helps confirmed that Tat was increases ΔΨₘ.

FIG. 5 shows that neuronal ATP/ADP ratio increases then declines with chronic Tat exposure. Primary cortical neurons were treated for 1, 24, or 48 hours with 0 (control), 0.1, or 2.5 μg/ml Tat. ATP levels, as well as ATP/ADP ratios, were sharply increased by both doses of Tat at 1 hour, and by 2.5 μg/ml Tat at 24 hours. By 48 hours, ATP levels, while still elevated, had declined from peak levels at 24 hours, and ATP/ADP ratio had fallen to control level or below (i.e. ≦1.0). P-values shown are versus time matched control.

FIG. 6 shows that the KATP channel antogonist Tolbutamide attenuates Tat-induced increases in ΔΨₘ and neuronal apoptosis. Co-treatment of 0.1 μg/ml or 2.5 μg/ml Tat treated cortical cultures with the mitochondrial KATP channel antagonist tolbutamide (100 μM) (A) completely prevented the Tat mediated rise in ΔΨₘ at 1 and 24 hours, and (B) partially attenuated Tat's dose-dependent induction of apoptotic cell death over 24 hours. 100 μM tolbutamide alone had no significant effect on ΔΨₘ, and showed no toxicity.

FIG. 7 shows that sublethal cPAF exposure leads to dendrite beading and disruption of spines. (A) cPAF causes dendrite beading and loss of dendritic spines. (A′) Low-magnification images show minimal changes in dendrite morphology and total spine number in control cultures (left). In cPAF-treated cultures (right), dendrite beading (red arrows) is accompanied by spine loss and sprouting of filopodia (green arrowheads) but gross aspects of dendrite structure are preserved. (B) No cells developed dendritic swellings in control cultures, while 55±3% of cPAF-exposed cells beaded. (C) 43±5% of dendritic spines were lost during cPAF exposure, while total spine number was maintained in control cultures.

FIG. 8 shows that cPAF promotes dendrite beading and failure of long-term potentiation in acute hippocampal slices. CA1 pyramidal neurons were exposed to 1 μM cPAF or vehicle for 30-60min and simultaneously imaged and recorded by whole-cell patch clamp before and after delivery of a high-frequency stimulus (HFS: three 1s, 100 Hz trains, every 20 s) applied to Schaffer collateral axons via a bipolar stimulating electrode planted in the stratum radiatum. (A) Dendrite beading in a cPAF-exposed cell 45min after HFS, with no disruption of dendrite or spine morphology in control cells. (B) HFS elicited beading in half of the cells from cPAF-treated slices, and in none from control slices. (C) Excitatory synaptic transmission between Shaffer collateral axons and CA1 pyramidal neurons is strongly potentiated following HFS in control slices. In cPAF-treated slices, EPSPs in cells that did not develop dendrite beading underwent a smaller potentiation, while EPSPs in cells whose dendrites did bead were not potentiated at all. (EPSP traces at baseline and 40 min from representative control cell and cPAF-treated beaded cell are at right.)

FIG. 9 shows that an adenosine receptor 2A (A_{2A}R) antagonist protects neurons against Tat-induced apoptosis. CGNs were exposed to HIV-1 Tat (Tat, 500 nM), or vehicle alone (NT), in the presence or absence of the A_{2A} antagonist, ZM241385. After 48 h, cultures were analyzed for the percentage of apoptotic cells using the TUNEL assay. Data represent mean±SEM for one experiment that was performed in triplicate; data are representative of two independent experiments.

FIG. 10 shows that ATL455, an A_{2A}R antagonist protects neurons against Tat-induced apoptosis. CGNs were exposed to HIV-1 Tat (Tat, 500 nM), or vehicle alone (No Tat), in the presence or absence of the A_{2A}R antagonists, ATL455 and ZM241385, or the A_{2A}R agonists ATL313 and CGS21680. After 48 h, cultures were analyzed for the percentage of apoptotic cells using the TUNEL assay. Data represent mean±SEM for one experiment that was performed in triplicate; data are representative of two independent experiments.

FIG. 11 shows that adenosine receptors control nitric oxide (NO) secretion induced by Tat. Human primary monocytes were treated with Tat in the presence or absence of the indicated adenosine receptor agonists for 8 h, and nitric oxide levels in the culture medium of the cells were then quantitated using the Griess reaction (Active Motif). Data represent mean+SEM of three experiments.

FIG. 12 shows that Tat-induced monocyte activation is opposed by the A_{2A}R agonist, CGS21680. Human primary monocytes were treated with Tat (100 nM) in the presence or absence of the A_{2A}R agonist, CGS21680 (CGS; 1 μM) or the A_{2A}R antagonist, ZM241385 (100 nM) for 4 h. TNF levels in culture supernatants were then quantitated by ELISA. Data represent mean+SEM of three replicates, from a single representative experiment

FIG. 13 shows Tat induces inflammatory gene expression by primary human monocytes. Primary monocytes were seeded in separate triplicate wells, and exposed independently to LPS (100 ng/ml), vehicle (NT) or Tat (100 nM) in the presence or absence of the A_{2A}R agonist, CGS21680 (1 μM). After 4 h, cells were harvested. (A) TNF was measured in cell supernatants by ELISA Mean data values (±S.D.) are shown for the triplicate wells. (B) The corresponding cells from each well in panel A were lysed and subjected to qRTPCR analysis. Each cDNA sample was analyzed in quadruplicate. Assay results are presented as fold induction of gene expression, compared to untreated cells, after normalization to levels of GAPDH. Data represent mean±SD for each individual experimental replicate/well.

FIG. 14 shows that Tat-induced monocyte activation is opposed by the A_{2A}R agonist, ATL313. Human primary monocytes were treated with Tat (100 nM) in the presence or absence of the A_{2A}R agonist, ATL313 (1-4 nM) or the A_{2A}R antagonist, ATL455 (1-4 nM) for 4 h. TNF levels in culture supernatants were then quantitated by ELISA. Data represent mean+SEM of three replicates, from a single representative experiment.

FIG. 15 shows a dose response analysis for inhibition of Tat-induced monocyte activation by the A_{2A}R agonist, ATL313. Human primary monocytes were treated with Tat (100 nM) in the presence or absence of the A_{2A}R agonist, ATL313 (at the indicated doses) for 4 h. TNF levels in culture supernatants were then quantitated by ELISA. Data represent mean+SEM of three replicates, from a single representative experiment.

### DETAILED DESCRIPTION OF THE INVENTION

The disclosed methods and compositions may be understood more readily by reference to the following detailed description of particular embodiments and the Example included therein and to the Figures and their previous and following description.

Provided are methods and compositions for treating HIV-related neurological disorders by inhibiting mitochondrial hyperpolarization. Thus, disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a hyperpolarization inhibitor is disclosed and discussed and a number of modifications that can be made to the hyperpolarization inhibitor are discussed, then each and every combination and permutation of the hyperpolarization inhibitor and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

It must be noted that as used herein and in the appended claims, the singular forms “a”, “an”, and “the” include plural reference unless the context clearly dictates otherwise. Thus, for example, reference to “a molecule” includes a plurality of such molecules, reference to “the molecule” is a reference to one or more molecules and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

### Methods of Treating and Preventing HIV Neurotoxin-Induced Neural Damage

Provided herein is a method of protecting a neuron from dysfunction induced by an HIV neurotoxin comprising contacting the cell with a therapeutically effective dose of an inhibitor of mitochondrial hyperpolarization. Non-limiting examples of neuronal dysfunction include alterations in: mitochondrial function and/or energy production; neurotransmitter uptake, recycling, and release; neuronal architecture; synaptic transmission, communication, and receptor dynamics; biochemical and signal transduction pathways, and neuronal cell death. Thus, in one aspect, an HIV neurotoxin can result in neuronal cell death. As used herein, neuronal cell death includes apoptosis, necrosis, or other non-specific death of neurons that can occur as a result of exposure to neurotoxins associated with HIV.

As used herein, apoptosis refers to programmed cell death that is signaled by the nuclei when age or state of cell health and condition dictates. Apoptosis is an active process requiring metabolic activity by the dying cell, often characterized by cleavage of the DNA into fragments that give a so called laddering pattern on gels. Cells that die by apoptosis do not usually elicit the inflammatory responses that are associated with necrosis. As used herein, necrosis refers to cell death in response to a major insult, resulting in a loss of membrane integrity, swelling and rupture of the cell. During necrosis, the cellular contents are released uncontrolled into the cell's environment which results in damage of surrounding cells and a strong inflammatory response in the corresponding tissue.

Disclosed is a method of treating or preventing HIV-1 associated dementia (HAD) in a subject in need of such treatment or prevention, comprising administering to the subject a therapeutically effective dose of an inhibitor of mitochondrial hyperpolarization. HIV associated dementia (HAD) is comprised of a spectrum of conditions from the mild HIV-1 minor cognitive-motor disorder (MCMD) to severe and debilitating AIDS dementia complex. Symptoms begin with motor slowing and may progress to severe loss of cognitive function, loss of bladder and bowel control, and paraparesis. A classification system has been formulated for HIV associated dementia, wherein subjects are classified as being Stage 0 (Normal), Stage 0.5 (Subclinical or Equivocal), Stage 1 (Mild), Stage 2 (Moderate), Stage 3 (Severe), or Stage 4 (End-Stage).Thus, the subject of the provided method can therefore be classified as Stage 0, Stage 0.5, Stage 1, Stage 2, Stage 3, or Stage 4.

By “treat” or “treatment” is meant a method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. For example, a disclosed method for treatment of HAD is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. For example, in the case of HAD, to treat HAD in a subject can comprise improving the disease classification. (e.g. from stage 3 to stage 2, from stage 2 to stage 1, from stage 1 to 0.5 or from stage 0.5 to 0).

As used throughout, “preventing” means to preclude, avert, obviate, forestall, stop, or hinder something from happening, especially by advance planning or action. For example, to prevent HAD in a subject is to stop or hinder the subject from advancing in disease classification (e.g. from stage 0 to stage 0.5, from stage 0.5 to stage 1, from stage 1 to stage 2, from stage 2 to stage 3, or from stage 3 to stage 4).

Microglia, macrophages and astrocytes are major HIV-1 targets in the brain, whereas HIV-1 infected neurons have been rarely observed. This indicates that indirect mechanisms may account for the severe neuronal damage observed in these patients. In addition to the production of cytokines, HIV-1 infected and/or functionally activated mononuclear cells and astrocytes can produce a number of soluble mediators, including the structural and regulatory proteins gp120, Tat, and platelet activating factor (PAF), which can exert damaging effects on both developing and mature neural tissues.

The HIV neurotoxin Tat, and its downstream mediator PAF [Perry, S. W., et al., J Biol Chem, 1998. 273(28): p. 17660-4] both affect synaptic activity. Tat provokes cell membrane depolarization and action potentials in neuronal cultures [Magnuson, D. S., et al., Ann Neurol, 1995. 37(3): p. 373-80] [Cheng, J., et al., Neuroscience, 1998. 82(1): p. 97-106], and PAF increases miniature synaptic activity and augments neurotransmitter release [Bouron, A., Prog Neurobiol, 2001. 63(6): p. 613-35]. Both Tat and PAF also induce mitochondrial hyperpolarization as described herein. Tumor necrosis factor-alpha (TNF-α) has also been shown to have synaptic effects [Beattie, E. C., et al., Science, 2002. 295(5563): p. 2282-85], and causes mitochondrial hyperpolarization as described herein. Other downstream mediators of Tat, PAF, or TNF-α, and other HIV neurotoxins, that do or may induce mitochondrial hyperpolarization include the cytokines interleukin-1 beta (IL-1μ), interleukin 6 (IL-6), interferon gamma (IFN-γ), the viral gene product gp120, and the signaling molecules arachidonic acid, cysteine, and nitric oxide. Thus, the neurotoxin of the present method can be PAF, TNF-α or Tat, or not exclusively, any of their downstream mediators or other HIV neurotoxins listed above

As used herein, mitochondrial hyperpolarization (MHP) refers to an elevation in the mitochondrial transmembrane potential, ΔΨₘ (delta psi), i.e., negative inside and positive outside). The ΔΨₘ is the result of an electrochemical gradient maintained by two transport systems—the electron transport chain and the F₀F₁-ATPase complex. For a review, see Perl et al. 2004 Trends in Immunol. 25:360-367. Briefly, the electron transport chain catalyzes the flow of electrons from NADH to molecular oxygen and the translocation of protons across the inner mitochondrial membrane, thus creating a voltage gradient with negative charges inside the mitochondrial matrix. F₀F₁-ATPase utilizes the extruded proton to synthesize ATP. MHP leads to uncoupling of oxidative phosphorylation, which disrupts ΔΨₘ and damages integrity of the inner mitochondrial membrane. Disruption of ΔΨₘ has been proposed as the point of no return in cell death signaling. This releases cytochrome c and other cell-death-inducing factors from mitochondria into the cytosol. Thus, the inhibitor of the present method can be a F₀F₁-ATPase agonists.

KATP channels participate in controlling plasma and mitochondrial membrane polarity, by controlling K⁺ efflux at the plasma membrane, and K⁺/H⁺ exchange at the mitochondrial membrane. As such, both plasma membrane and mitochondrial membrane KATP channels can effect mitochondrial polarization. Thus, the inhibitor of the present method can be a KATP channel antagonist. The KATP channel antagonist can be selected from the group consisting of Tolbutamide, hydroxydecanoic acid (5-HD), glibenclamide (glyburide), and meglitinide analog (e.g. Repaglinide, A-4166).

The inhibitor of the present method can be an electron transport inhibitor. The electron transport chain (ETC) is the biomolecular machinery present in mitochnodria that couples the flow of electrons to proton pumps in order to convert energy from sugar to ATP. The electron transport chain couples the transfer of an electron from NADH (nicotinamide adenine dinucleotide) to molecular oxygen (O₂) with the pumping of protons (H+) across a membrane. The charge gradient that results across the membrane serves as a battery to drive ATP Synthase. The electron transport chain is made up of several integral membrane complexes: NADH dehydrogenase (complex 1), Coenzyme Q—cytochrome c reductase (complex III), and Cytochrome c oxidase (complex IV). Succinate —Coenzyme Q reductase (Complex II) connects the Krebs cycle directly to the electron transport chain.

Thus, the inhibitor of the provided method can be an inhibitor of any component of the ETC. Thus, the inhibitor can be an inhibitor of complex I, II, III, or IV. For example, diphenylene iodonium (DPI) and rotenone are specific inhibitors of complex I, succinate-q reductase (TTFA) is an inhibitor of complex II, antimycin A and myxothiazole are inhibitors of complex III, and potassium cyanide (KCN) is an inhibitor of complex IV. Thus, the inhibitor of the provided method can be selected from the group consisting of diphenylene iodonium (DPI), rotenone, antimycin, myxothiazole, succinate-q reductase (TTFA), and potassium cyanide (KCN).

The inhibitor of the present method can be an uncoupler. As used herein an “uncoupler” is a substance that allows oxidation in mitochondria to proceed without the usual concomitant phosphorylation to produce ATP; these substances thus “uncouple” oxidation and phosphorylation. As an example, Trifluorocarbonylcyanide Phenylhydrazone (FCCP) is a chemical uncoupler of electron transport and oxidative phosphorylation. FCCP permeabilizes the inner mitochondrial membrane to protons, destroying the proton gradient and, in doing so, uncouples the electron transport system from the oxidative phosphorylation system. In this situation, electrons continue to pass through the electron transport system and reduce oxygen to water, but ATP is not synthesized in the process.

The uncoupler of the present method can agonize, antagonize or modulate the expression of endogenous mitochondrial uncoupling proteins (UCPs). As a non-limiting example, the uncoupler of the present method can be the beta-adrenergic agonist CL-316,243 (disodium (R,R)-5-(2-((2-(3-chlorophenyl)-2-hydroxyethyl)-amino)propyl)-1,3-benzodioxole-2,3-dicarboxylate) (Yoshida et. al., Am J Physiol. 1998. 274(3 Pt 1): p. E469-75).

The uncoupler of the present method can be a protonophore. Thus, the inhibitor of the present method can be a protonophore. As used herein, a “protonophore” is a molecule that allows protons to cross lipid bilayers. The protonophore can be FCCP. The protonophore can also be 2,4,-dinitrophenol (DNP). The protonophore can be also m-chlorophenylhydrazone (CCCP). The protonophore can also be pentachlorophenol (PCP).

The disclosed method can further comprise contacting the cell with an antioxidant. Generally, antioxidants are compounds that react with, and typically get consumed by, oxygen. Since antioxidants typically react with oxygen, antioxidants also typically react with the free radical generators, and free radicals. (“The Antioxidants—The Nutrients that Guard Your Body” by Richard A. Passwater, Ph. D., 1985, Keats Publishing Inc., which is herein incorporated by reference at least for material related to antioxidants). The herein disclosed antioxidant can be any antioxidant, and a non-limiting list would included but not be limited to, non-flavonoid antioxidants and nutrients that can directly scavenge free radicals including multi-carotenes, beta-carotenes, alpha-carotenes, gamma-carotenes, lycopene, lutein and zeanthins, selenium, Vitamin E, including alpha-, beta- and gamma-(tocopherol, particularly α-tocopherol, etc., vitamin E succinate, and trolox (a soluble Vitamin E analog) Vitamin C (ascoribic acid) and Niacin (Vitamin B3, nicotinic acid and nicotinamide), Vitamin A, 13-cis retinoic acid, , N-acetyl-L-cysteine (NAC), sodium ascorbate, pyrrolidin-edithio-carbamate, and coenzyme Q10; enzymes which catalyze the destruction of free radicals including peroxidases such as glutathione peroxidase (GSHPX) which acts on H₂O₂ and such as organic peroxides, including catalase (CAT) which acts on H₂O₂, superoxide dismutase (SOD) which disproportionates 0₂H₂O₂ ; glutathione transferase (GSHTx), glutathione reductase (GR), glucose 6-phosphate dehydrogenase (G6PD), and mimetics, analogs and polymers thereof (analogs and polymers of antioxidant enzymes, such as SOD, are described in, for example, U.S. Pat. No. 5,171,680 which is incorporated herein by reference for material at least related to antioxidants and antioxidant enzymes); glutathione; ceruloplasmin; cysteine, and cysteamine (beta-mercaptoethylamine) and flavenoids and flavenoid like molecules like folic acid and folate. A review of antioxidant enzymes and mimetics thereof and antioxidant nutrients can be found in Kumar et al, Pharmac. Ther. Vol 39: 301, 1988 and Machlin L. J. and Bendich, F.A.S.E.B. Journal Vol.1:441-445, 1987 which are incorporated herein by reference for material related to antioxidants.

Thus, the disclosed method can further comprise contacting the cell with an antioxidant selected from the group consisting of tauroursodeoxycholic acid (TUDCA), N-acetylcysteine (NAC) (600-800 mg/day), Mito-Coenzyme Q10 (Mito-CoQ) (300-400 mg/day), Mito-VitaminE (Mito-E) (100-1000 mg/day), Coenzyme Q10 (300-400 mg/day), and idebenone (60-120 mg/day).

N-acetylcysteine (NAC) is used to replenish Glutathione (GSH) that has been depleted in HIV-infected individuals by acetaminophen overdose. (De Rosa S C, Zaretsky M D, Dubs J G, Roederer M, Anderson M, Green A, Mitra D, Watanabe N, Nakamura H, Tjioe I, Deresinski S C, Moore W A, Ela S W, Parks D, Herzenberg L A, Herzenberg L A. N-acetylcysteine replenishes glutathione in HIV infection. European Journal of Clinical Investigation, 30(10):915). Thus, in one embodiment of the provided invention, NAC is not used to replenish Glutathione (GSH) in HIV-infected subjects. In another embodiment of the method NAC is not used to treat HAD.

Coenzyme Q10 has been used to treat patients having the AIDS related complex. (Folkers K, Hanioka T, Xia L J, McRee J T Jr, Langsjoen P. Coenzyme Q10 increases T4/T8 ratios of lymphocytes in ordinary subjects and relevance to patients having the AIDS related complex. Biochem Biophys Res Commun. 1991 Apr. 30;176(2):786-91.) Bile acids such as TUDCA lead to a significant improvement in serum transaminase activities in subjects with hepatitis B and C. (Chen W, Liu J, Gluud C. Bile acids for viral hepatitis. Cochrane Database Syst Rev. 2003;(2):CD003181.) Thus, in one embodiment of the provided invention, Coenzyme Q10 is not used to treat patients having the AIDS related complex. In another embodiment of the method Coenzyme Q10 is not used to treat HAD.

Idebenone has been used to treat subjects with senile cognitive decline (Bergamasco B, Villardita C, Coppi R. Effects of idebenone in elderly subjects with cognitive decline. Results of a multicentre clinical trial. Arch Gerontol Geriatr. 1992 Nov.-Dec.; (3):279-86.).) Thus, in one embodiment of the provided invention, Idebenone not used to treat subjects with senile cognitive decline. In another embodiment of the method Idebenone is not used to treat HAD.

The disclosed method can further comprise contacting the cell with an antiretroviral compound. Antiretroviral drugs inhibit the reproduction of retroviruses such as HIV. Antiretroviral agents are virustatic agents which block steps in the replication of the virus. The drugs are not curative; however continued use of drugs, particularly in multi-drug regimens, can significantly slow disease progression. There are three main types of antiretroviral drugs, although only two steps in the viral replication process are blocked. Nucleoside analogs, or nucleoside reverse transcriptase inhibitors (NRTIs), act by inhibiting the enzyme reverse transcriptase. Because a retrovirus is composed of RNA, the virus must make a DNA strand in order to replicate itself. Reverse transcriptase is an enzyme that is essential to making the DNA copy. The nucleoside reverse transcriptase inhibitors are incorporated into the DNA strand. This is a faulty DNA molecule that is incapable of reproducing. The non-nucleoside reverse transcriptase inhibitors (NNRTIs) act by binding directly to the reverse transcriptase molecule, inhibiting its activity. Protease inhibitors act on the enzyme protease, which is essential for the virus to break down the proteins in infected cells. Without this essential step, the virus produces immature copies of itself, which are non-infectious. A fourth class of drugs called fusion inhibitors block HIV from fusing with healthy cells.

Thus, the antiretroviral compound can comprise one or more molecules selected from the group consisting of protease inhibitors [PI], fusion inhibitors, nucleoside reverse transcriptase inhibitors [NRTI], and non-nucleoside reverse transcriptase inhibitors [NNRTI].

Thus, the antiretroviral compound of the provided method can be a PI, such as a PI selected from the group consisting of Indinavir, Amprenavir, Nelfinavir, Saquinavir, Fosamprenavir, Lopinavir, Ritonavir, and Atazanavir, or any combinations thereof.

Thus, the antiretroviral compound of the provided method can be a fusion inhibitor, such as for example Enfuvirtide.

Thus, the antiretroviral compound of the provided method can be a NRTI, such as a NRTI selected from the group consisting of Abacavir, Stavudine, Didanosine, Lamivudine, Zidovudine, Zalcitabine, Tenofovir, and Emtricitabine, or any combinations thereof.

Thus, the antiretroviral compound of the provided method can be a NNRTI, such as a NNRTI selected from the group consisting of Efavirenz, Nevirapine, and Delavirdine. The disclosed method can further comprise administering to the subject a neurotoxin inhibitor. The inhibitor can be a TNFα inhibitor, including TNFα-inhibitory monoclonal antibodies (e.g., etanercept), phosphodiesterase (PDE)-4 inhibitors (such as IC485, which can reduce TNFα production), thalidomide and other agents.

Etanercept is a dimeric fusion protein consisting of the extracellular ligand-binding portion of the human 75 kilodalton (p75) tumor necrosis factor receptor (TNFR) linked to the Fc portion of human IgG1. The Fc component of etanercept contains the CH2 domain, the CH3 domain and hinge region, but not the CHI domain of IgG1. Etanercept is produced by recombinant DNA technology in a Chinese hamster ovary (CHO) mammalian cell expression system. It consists of 934 amino acids and has an apparent molecular weight of approximately 150 kilodaltons. Etanercept has been evaluated in HIV-infected subjects receiving highly active antiretroviral therapy (HAART) (Sha B E, Valdez H, Gelman R S, Landay A L, Agosti J, Mitsuyasu R, Pollard R B, Mildvan D, Namkung A, Ogata-Arakaki D M, Fox L, Estep S, Erice A, Kilgo P, Walker R E, Bancroft L, Lederman M M. Effect of etanercept (Enbrel) on interleukin 6, tumor necrosis factor alpha, and markers of immune activation in HIV-infected subjects receiving interleukin 2. AIDS Res Hum Retroviruses. 2002 Jun. 10;18(9):661-5).

IC485 is an orally administered, small molecule inhibitor of PDE4. inhibition of PDE4 leads to an increase in the second messenger, cAMP, within cells. This inhibition may in turn reduce the cell's production of tumor necrosis factor alpha (TNF-alpha) and a variety of other inflammatory mediators. IC485 is being evaluated in patients with chronic obstructive pulmonary disease.

The inhibitor can be a PAF receptor antagonist (such as lexipafant, WEB2086, WEB2170, BN-52021 or PMS-601), a PAF degrading-enzyme such as PAF-acetylhydrolase (PAF-AH), or a molecule that regulates the expression of PAF-AH (such as pioglitazone and other PPAR-gamma inhibitors).

Lexipafant has been used improve cognitive dysfunction in HIV-infected people (Schifitto G, Sacktor N, Marder K, McDermott M P, McArthur J C, Kieburtz K, Small S, Epstein L G. Randomized trial of the platelet-activating factor antagonist lexipafant in HIV-associated cognitive impairment. Neurological AIDS Research Consortium. Neurology. 1999 Jul. 22;53(2):391-6). Lexipafant can be administered at for example 500 mg/day.

PMS-601 inhibits proinflammatory cytokine synthesis and HIV replication (Martin M, Serradji N, Dereuddre-Bosquet N, Le Pavec G, Fichet G, Lamouri A, Heymans F, Godfroid J J, Clayette P, Dormont D. PMS-601, a new platelet-activating factor receptor antagonist that inhibits human immunodeficiency virus replication and potentiates zidovudine activity in macrophages. Antimicrob Agents Chemother. 2000 November;44(11):3150-4.)

TNF-alpha-mediated neuronal apoptosis can also be blocked by co-incubation with PAF acetylhydrolase (PAF-AH) (Perry S W, Hamilton J A, Tjoelker L W, Dbaibo G, Dzenko K A, Epstein L G, Hannun Y, Whittaker J S, Dewhurst S, Gelbard H A. Platelet-activating factor receptor activation. An initiator step in HIV-1 neuropathogenesis. J Biol Chem. 1998 Jul. 10;273(28):17660-4).

Pioglitazone can inhibit PAF-induced morphological changes through PAF-AH (Sumita C, Maeda M, Fujio Y, Kim J, Fujitsu J, Kasayama S, Yamamoto I, Azuma J. Pioglitazone induces plasma platelet activating factor-acetylhydrolase and inhibits platelet activating factor-mediated cytoskeletal reorganization in macrophage. Biochim Biophys Acta. 2004 Aug. 4; 1673(3):115-21).

Phosphatidylcholines (1-O-alcoxy-2-amino-2-desoxy-phosphocholines and 1-pyrene-labeled analogs) were synthesized and used to examine interactions with recombinant human PAF-AH (Deigner H P, Kinscherf R, Claus R, Fymys B, Blencowe C, Hermetter A. Novel reversible, irreversible and fluorescent inhibitors of platelet-activating factor acetylhydrolase as mechanistic probes. Atherosclerosis. 1999 May; 144(1):79-90).

The disclosed method can further comprise administering to the subject an inhibitor of GSK-3β. The inhibitor can be valproate or lithium.

Valproate has been administered to HIV-infected patients receiving efavirenz or lopinavir (DiCenzo R, Peterson D, Cruttenden K, Morse G, Riggs G, Gelbard H, Schifitto G. Effects of valproic acid coadministration on plasma efavirenz and lopinavir concentrations in human immunodeficiency virus-infected adults. Antimicrob Agents Chemother. 2004 November;48(11):4328-31). A typical dose of valproate comprises 250 mg twice daily.

The disclosed method can further comprise administering to the subject a compound that enhances CNS uptake. Ritonavir influences levels of coadministered drugs in the CNS, due to effects on the activity of drug transporters located at the BBB (Haas D W, Johnson B, Nicotera J, Bailey V L, Harris V L, Bowles F B, Raffanti S, Schranz J, Finn T S, Saah A J, Stone J Effects of ritonavir on indinavir pharmacokinetics in cerebrospinal fluid and plasma Antimicrob Agents Chemother. 2003 July;47(7):2131-7).

The disclosed methods can further comprise administering a drug that inhibits the P-glycoprotein drug efflux pump, or multidrug resistance-associated proteins at the blood-brain-barrier (BBB). These include LY-335979 (Choo E F, Leake B, Wandel C, Imamura H, Wood A J, Wilkinson G R, Kim R B. Pharmacological inhibition of P-glycoprotein transport enhances the distribution of HIV-1 protease inhibitors into brain and testes. Drug Metab Dispos. 2000 June;28(6):655-60) and PSC-833 and GF120918 (Pgp blockers) (Polli J W, Jarrett J L, Studenberg S D, Humphreys J E, Dennis S W, Brouwer K R, Woolley J L. Role of P-glycoprotein on the CNS disposition of amprenavir (141W94), an HIV protease inhibitor. Pharm Res. 1999 August;16(8):1206-12; Kemper E M, van Zandbergen A E, Cleypool C, Mos H A, Boogerd W, Beijnen J H, van Tellingen O. Increased penetration of paclitaxel into the brain by inhibition of P-Glycoprotein. Clin Cancer Res. 2003 July;9(7):2849-55) as well as MK571 (a specific Mrp family inhibitor):

The disclosed method can further comprise administering to the subject a microglial deactivator. Minocyclin is a potent microglial deactivator (Wu DC, Jackson-Lewis V, Vila M, Tieu K, Teismann P, Vadseth C, Choi D K, Ischiropoulos H, Przedborski S. Blockade of microglial activation is neuroprotective in the 1-methyl-4-phenyl-1,2,3,6-tetrahydropyridine mouse model of Parkinson disease. J Neurosci. 2002 Mar. 1;22(5):1763-71; Yijanheikki J, Keinanen R, Pellikka M, Hokfelt T, Koistinaho J. Tetracyclines inhibit microglial activation and are neuroprotective in global brain ischemia. Proc Natl Acad Sci U S A. 1998 Dec. 22;95(26):15769-74). Further, minocycline can potently inhibit HIV-1 viral production from microglia (Si Q, Cosenza M, Kim M O, Zhao M L, Brownlee M, Goldstein H, Lee S. A novel action of minocycline: inhibition of human immunodeficiency virus type 1 infection in microglia. J Neurovirol. 2004 October; 10(5):284-92). Thus, the microglial deactivator can be minocycline. A typical dosage of minocyclin comprises 200 mg/day.

Other microglial deactivators that can be used in the present methods include PDE4 inhibitors (described above).

The disclosed method can further comprise administering to the subject an inhibitor of glutamate damage. The inhibitor can be a beta-lactam antibiotic such as for example ceftriaxone, which can have direct effects on glutamate transporter expression.

When delivered to animals, the beta-lactam ceftriaxone increases both brain expression of GLT 1 that inactivates synaptic glutamate (Rothstein J D, Patel S, Regan M R, Haenggeli C, Huang Y H, Bergles D E, Jin L, Dykes Hoberg M, Vidensky S, Chung D S, Toan S V, Bruijn L I, Su Z Z, Gupta P, Fisher P B. Beta-lactam antibiotics offer neuroprotection by increasing glutamate transporter expression. Nature. 2005 Jan. 6;433(7021):73-7) A typical dosage of cephtriaxone is 50 mg/kg/day.

A dose-dependent inhibition of high affinity glutamate uptake sites is observed after addition of exogenous recombinant human TNFα to human fetal astrocytes (PHFAs) (Fine S M, Angel R A, Perry S W, Epstein L G, Rothstein J D, Dewhurst S, Gelbard H A. Tumor necrosis factor alpha inhibits glutamate uptake by primary human astrocytes. Implications for pathogenesis of HIV-1 dementia. J Biol Chem. 1996 Jun. 28;271(26):15303-6). Thus, the inhibitor of glutamate damage can be a TNFβ inhibitor or a microglial deactivator (described above), which can have indirect effects on glutamate transporters.

The disclosed method can further comprise administering to the subject a therapeutically effective dose of a modulator of adenosine receptor signaling. Endogenous adenosine plays a pivotal role in the regulation of neural cell fate. The actions of adenosine are mediated by specific receptors located on cell membranes, which belong to the family of G protein-coupled receptors. Currently, four adenosine receptors have been cloned: A₁, A_{2A}, A_{2B}, and A₃. The disclosed modulator of adenosine receptor signaling can comprise any composition that will alter a biological property of either adenosine or adenosine receptors in a cell, such as for example their synthesis, degradation, translocation, binding, or phosphorylation, such that the alteration results in a net increase or decrease in adenosine receptor signaling in the cell. As a non-limiting example, the provided modulator can be a nucleic acid that alters expression of either adenosine or adenosine receptor in a cell, such as for example RNAi or antisense nucleic acids. As another example, the provided modulator can be a polypeptide that alters the binding of adenosine to adenosine receptors, such as for example soluble adenosine receptors, mutant adenosine ligands or antibodies specific for adenosine or adenosine receptors. As another example, the provided modulator can comprise informational molecules that modulate adenosine receptor expression (such as short-interfering RNAs or peptide nucleic acids) or molecules that may regulate downstream signaling events that may occur as a result of adenosine receptor stimulation.

Thus, the provided modulator of adenosine receptor signaling can be a small molecule comprising a modified adenosine (6-amino-9-beta-D-ribofuranosyl-9-H-purine). Modifications that can be made to adenosine are well known in the art. These modifications include those that result in adenosine receptor agonists and antagonists. These agonists and antagonists can be either receptor selective or non-selective. Provided herein is the use of these adenosine receptor agonists and antagonists in the treatment of HAD.

The modulator of the present method can be an adenosine 1 receptor (A₁R) antagonist. The modulator can be an adenosine 2A receptor (A_{2A}R) antagonist. The modulator can be an adenosine 2B receptor (A_{2B}R) antagonist. The modulator can be an adenosine 3 receptor (A₃R) antagonist. Thus, the modulator can be any adenosine receptor selective antagonist, whether known in the art or later developed. Non-limiting examples of A_{2A}R selective antagonists include ATL455, ZM241385, KW-6002 (istradefylline), SCH 58261, and the pharmaceutically acceptable salts thereof.

ZM241385 is 4(2-[7-Amino-2-(2-furyl)[1,2,4]triazolo[2,3-a][1,3,5]triazin-5-ylamino]ethyl)phenol (Poucher et al. (1995) The in vitro pharmacology of ZM 241385, a potent, non-xanthine, A₂ₐ selective adenosine receptor antagonist. Br. J. Pharmacol. 115 1096; Poucher et al (1996) Pharmacodynamics of ZM 241385, a potent A₂ₐ adenosine receptor antagonist, after enteric administration in rat, cat and dog. J. Pharm. Pharmacol. 48 601; Keddie et al (1996) In vivo characterisation of ZM 241385, a selective adenosine A_{2A} receptor antagonist. Eur. J. Pharmacol. 301 107.)

KW-6002 (istradefylline) is (E)-1,3-diethyl-8-(3,4-dimethoxystyryl)-7-methyl-3,7-dihydro-1H-purine-2,6-dione. KW-6002 has been evaluated humans as a treatment for Parkinson's disease (W. Bara-Jimenez, M D, A. Sherzai, M D, T. Dimitrova, M D, A. Favit, M D, F. Bibbiani, M D, M. Gillespie, N P, M. J. Morris, M R C Psych, M. M. Mouradian, M D and T. N. Chase, MD Adenosine A_{2A} receptor antagonist treatment of Parkinson's disease. Neurology. 2003 Aug. 12;61(3):293-6).

SCH 58261 is 7-(2-phenylethyl)-5-amino-2-(2-furyl)-pyrazolo-[4,3-e]-1,2,4-triazolo[1,5-c]pyrimidine.

These modifications to adenosine to produce antagonists are exemplary and provide guidance to and description for other antagonistic adenosine modifications.

The provided modulator can be an adenosine 1 receptor (A₁R) agonist. The modulator can be an adenosine 2A receptor (A_{2A}R) agonist. The modulator can be an adenosine 2B receptor (A_{2B}) agonist. The modulator can be an adenosine 3 receptor (A₃R) agonist, such as for example CF101 (Aderis Pharmaceuticals). Thus, the provided modulator can be any adenosine receptor selective agonist, whether known in the art or later developed. Non-limiting examples of A_{2A}R selective agonist include ATL146e, ATL313, PJ-1165, Binodenoson (MRE-0470), MRE-0094, CGS21680, and the pharmaceutically acceptable salts thereof.

ATL146e is 4-{3-[6-amino-9-(5-ethylcarbamoyl-3,4-dihydroxytetrahydrofuran-2-yl)-9H-purin-2-yl]prop-2-ynyl}cyclohexanecarboxylic acid methyl ester. (Lappas C M, et al. A_{2A} adenosine receptor induction inhibits IFN-gamma production in murine CD4+ T cells. J Immunol. 2005 Jan. 15;174(2):1073-80.)

ATL313 is 4-{3-[6-amino-9-(5-cyclopropylcarbamoyl-3,4-dihydroxytetrahydrofuran-2-yl)-9H-purin-2-yl]prop-2-ynyl}piperidine-1-carboxylic acid methyl ester (Lappas C M, et al. A_{2A} adenosine receptor induction inhibits IFN-gamma production in murine CD4+ T cells. J Immunol. 2005 Jan. 15;174(2):1073-80.)

CGS21680 is 4-[2-[[6-Amino-9-(N-ethyl-b-D-ribofuranuronamidosyl)-9H-purin-2yl]amino]ethyl]benzenepropanoic acid hydrochloride. (Phillis et al (1990) The selective adenosine A₂ receptor agonist, CGS 21680, is a potent depressant of cerebral cortical neuronal activity. Brain Res. 509 328. Nekooeian and Tabrizchi (1998) Effects of CGS 21680, a selective A_{2A} adenosine receptor agonist, on cardiac output and vascular resistance in acute heart failure in the anaesthetized rat. Br. J. Pharmacol. 123 1666. Klotz (2000) Adenosine receptors and their ligands. Naunyn-Schmied. Arch. Pharmacol. 362-382).

These modifications to adenosine to produce agonists are exemplary and provide guidance to and description for other agonistic adenosine modifications.

Any of the compounds described herein can be the pharmaceutically-acceptable salt thereof. In one aspect, pharmaceutically-acceptable salts are prepared by treating the free acid with an appropriate amount of a pharmaceutically-acceptable base. For example, one or more hydrogen atoms of the SO₃H group can be removed with a base. Representative pharmaceutically-acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like.

In another aspect, if the compound possesses a basic group, it can be protonated with an acid such as, for example, HCl or H₂SO₄, to produce the cationic salt. For example, the techniques disclosed in U.S. Pat. No. 5,436,229 for producing the sulfate salts of argininal aldehydes, which is incorporated by reference in its entirety, can be used herein. In one aspect, the reaction of the compound with the acid or base is conducted in water, alone or in combination with an inert, water-miscible organic solvent, at a temperature of from about 0 C to about 100 C such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

It is contemplated that the pharmaceutically-acceptable salts of the compounds described herein can be used as prodrugs or precursors to the active compound prior to the administration. For example, if the active compound is unstable, it can be prepared as its salt form in order to increase stability in dry form (e.g., powder).

The severity of dementia in persons with HIV-1 associated neurologic disease is strongly correlated with the number of macrophages and microglia within the basal ganglia and frontal lobes [Glass, J. D., et al. 1995. Ann Neurol 38:755-762]. Thus, the activation of microglia and brain macrophages plays a crucial role in the induction of neuronal dysfunction and damage. Thus, the herein disclosed agonists of adenosine receptor signaling can inhibit HAD in a subject in part by inhibiting the recruitment of monocytes to the CNS.

### Screening Methods

Disclosed is a method of identifying a compound that can promote neural cell protection, the method comprising contacting a neural cell with a candidate neural protecting compound, contacting the neural cell with an agent that induces mitochondrial hyperpolarization, and evaluating the ability of the compound to prevent or inhibit mitochondrial hyperpolarization in the cell. The agent of the provide method can be a neurotoxin. The neurotoxin can be an HIV neurotoxin. The neurotoxin can be Tat or platelet-activating factor (PAF), or an analog thereof. Thus, the neurotoxin can be carbamyl-platelet-activating factor (c-PAF).

In the disclosed method, a decrease in vesicle recycling, mitochondrial membrane potential, ATP/ADP ratios, NADH/NAD+ ratios, reactive oxygen species (ROS), dendritic beading, and cell death can indicate inhibition of hyperpolarization in the cell.

Vesicle recycling can be evaluating in a cell by monitoring vesicular uptake and release of the styryl dye FM1-43 as described herein.

Mitochondrial membrane potential can be evaluated in a cell by monitoring mitochondrial accumulation of cationic dyes such as, but not exclusively, tetramethylrhodamine ethyl and methyl esters (TMRE and TMRM respectively), which accumulate in mitochondria proportional to the magnitude of the membrane potential.

ATP/ADP ratios can be evaluated in a cell by measuring ATP levels by the luminescent luciferase reaction, then measuring ADP levels by biochemical reactions that convert ADP to ATP, whereby the difference in luminescence signal pre- and post-ADP conversion represents the ADP levels of the culture. Results are then expressed as an ATP/ADP ratio.

NADH/NAD⁺ ratios can be evaluated in a cell by measuring autofluorescence of NADH/NADPH in the cell, whereby a decrease in autofluorescence indicates increased reduction of NADH/NADPH to NAD+/NADP+, and hence a lower NADH/NAD+ratio (and vice versa).

Reactive oxygen species can be evaluated in a cell by numerous methods, including but not limited to monitoring oxidation (i.e. increased fluorescence) of intracellularly accumulated 5-(and-6)-chloromethyl-2′,7′-dichlorodihydrofluorescein diacetate, acetyl ester (CM-H₂DCFDA), an oxidizable dye indicator

Dendritic beading can be evaluated in a cell by electrical stimulation of the cell, and counting the number of beaded region, as described herein.

Cell death, including apoptosis and necrosis, can be evaluated in a cell using standard methods known in the art. Apoptosis and cell mediated cytotoxicity are characterized by cleavage of the genomic DNA into discrete fragments prior to membrane disintegration. Because DNA cleavage is a hallmark for apoptosis, assays which measure prelytic DNA fragmentation are especially attractive for the determination of apoptotic cell death. These DNA fragments reveal, upon agarose gel electrophoresis, a distinctive ladder pattern consisting of multiples of an approximately 180 bp. Radioactive as well as non-radioactive methods to detect and quantify DNA fragmentation in cell populations have been developed. In general, these methods are based on the detection and/or quantification of either low molecular weight (LMW) DNA which is increased in apoptotic cells or high molecular weight (HMW) DNA which is reduced in apoptotic cells.

Further, proteases known as caspases are involved in the early stages of apoptosis. The appearance of these caspases sets off a cascade of events that disable a multitude of cell functions. Caspase activation can be analyzed by an in vitro enzyme assay. For example, activity of a specific caspase (e.g. caspase 3) can be determined in cellular lysates by capturing of the caspase and measuring proteolytic cleavage of a suitable substrate. Caspase activation can also be analyzed by detection of cleaved substrate. As an example, caspase 3's substrate PARP (Poly-ADP Ribose-Polymerase) and the cleaved fragments can be detected with the anti-PARP antibody. There are also commercially available antibodies that specifically bind, for example, active caspase-3 (i.e., cleaved procaspase-3).

Non-limiting examples of neural cells that can be used in the disclosed methods include neural cells from a primary cell culture of cerebellar granule neurons (CGNs), cortical neurons (CN), hippocampal neurons (HN), striatal neurons (SN), substantia nigra neurons (SN), or fetal neurons. Non-limiting examples of neural cell lines that might be used include PC-12 and SK-N-MC cell lines, and derivatives.

The candidate neural protecting molecule that can be identified by the method provided method can be from any of the following chemical classes: a KATP antagonist, an inhibitor of electron transport, a protonophore, or an antioxidant. The candidate compound of the provided method can be produced using standard chemical synthesis techniques. Thus, provided herein is a compound produced by the herein disclosed method(s).

### Hyperpolarization Inhibitory Compositions and Administration Methods

Disclosed herein is a composition, comprising a molecule that inhibits mitochondrial hyperpolarization in a neural cell and an antiretroviral compound. The hyperpolarization inhibitor of the composition can comprise one or more of a KATP antagonist, an inhibitor of electron transport, a protonophore, or an antioxidant. The antiretroviral compound can be any antiretroviral compound as disclosed herein, such as one or more molecules selected from the group consisting of protease inhibitors [PI], nucleoside reverse transcriptase inhibitors [NRTI], and non-nucleoside reverse transcriptase inhibitors [NNRTI].

### Therapeutic Doses

The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of the compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose.

The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. For example, the disclosed anti-retroviral compounds and antioxidants can be administered at published dosages, such as those approved for human use, e.g., in the treatment of HIV-1 infection.

A typical daily dosage of the disclosed inhibitors of hyperpolarization used alone can range from about 0.001 mg/kg to up to 50 mg/kg of body weight or more per day, depending on the factors mentioned above. In one aspect, the disclosed KATP channel antagonists can be administered at from 0.02 mg/kg to about 30 mg/kg of body weight per day. As non-limiting examples, Tolbutamide can be administered at from about 0.25 to 3 g/day; glibenclamide (glyburide) can be administered at from about 1.25 to 20 mg/day; and meglitinide analog (e.g. Repaglinide, A-4166) can be administered at from about 0.5 to 4 mg/day.

In another aspect, the disclosed inhibitors of the ECC (e.g., DPI, rotenone, antimycin, myxothiazole, TTFA, and KCN can be administered at from 0.001 mg/kg to 1 mg/kg of body weight per day. In another aspect, the disclosed protonophore (e.g., FCCP, DNP, CCCP, PCP) can be administered at from 0.001 mg/kg to 1 mg/kg of body weight per day. In one aspect, the disclosed beta-adrenergic agonist CL-316,243 can be administered at 0.01 to up to 1 mg/kg, including 0.1 mg/kg, of body weight or more per day.

In another aspect, the disclosed antioxidants can be administered at from 1 mg/day to 1000 mg/day. As non-limiting examples, N-acetylcysteine (NAC) can be administered at from about 600 mg/day to 800 mg/day; Mito-Coenzyme Q10 (Mito-CoQ) can be administered at from about 300 mg/day to 400 mg/day; Mito-VitaminE (Mito-E) can be administered from about 100 to 1000 mg/day); Coenzyme Q10 can be administered from about 300 mg/day to 400 mg/day; and idebenone can be administered at from about 60 mg/day to 120 mg/day.

A typical daily dosage of the disclosed modulators of adenosine receptor signaling used alone can range from about 0.05 to 5 mg/kg of body weight or more per day, depending on the factors mentioned above. In one aspect, the disclosed A_{2A}R antagonists (e.g. ATL455, KW6002 and ZM241685) can be administered at doses ranging from 0.3 to 3 mg/kg of body weight per day; KW6002 can be administered to humans at doses up to 40 mg/day. In another aspect, the disclosed A_{2A}R agonists (e.g. ATL146e, ATL313 and CGS21680) can be administered at from 0.05 to 50 mg/kg of body weight per day.

### Pharmaceutically Acceptable Carriers

The compositions can also be administered in vivo in a pharmaceutically acceptable carrier. By “pharmaceutically acceptable” is meant a material that is not biologically or otherwise undesirable, i.e., the material can be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. Thus, the disclosed compositions can be administered intracranially intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The compositions may be administered orally or parenterally (e.g., intravenously, intramuscular injection, by intraperitoneal injection, transdermally, extracorporeally, intracranially, topically or the like, including topical intranasal administration or administration by inhalant. As used herein, “intracranial administration” means the direct delivery of substances to the brain including, for example, intrathecal, intracisternal, intraventricular or trans-sphenoidal delivery via catheter or needle. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

The following examples are set forth below to illustrate the methods and results according to the present invention. These examples are not intended to be inclusive of all aspects of the present invention, but rather to illustrate representative methods and results. These examples are not intended to exclude equivalents and variations of the present invention which are apparent to one skilled in the art.

### EXAMPLES

### Example 1

### Bioenergetic Defects in Neurons Exposed to HIV-1 Neurotoxins

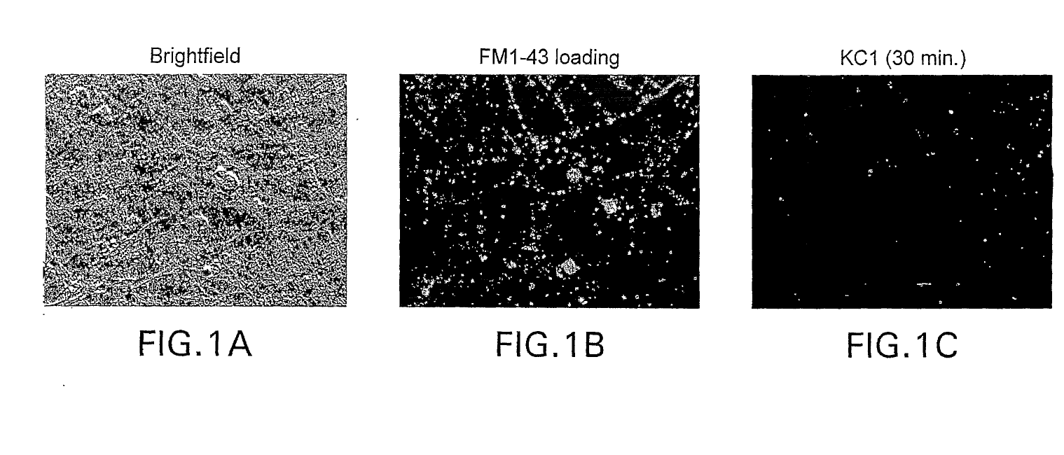

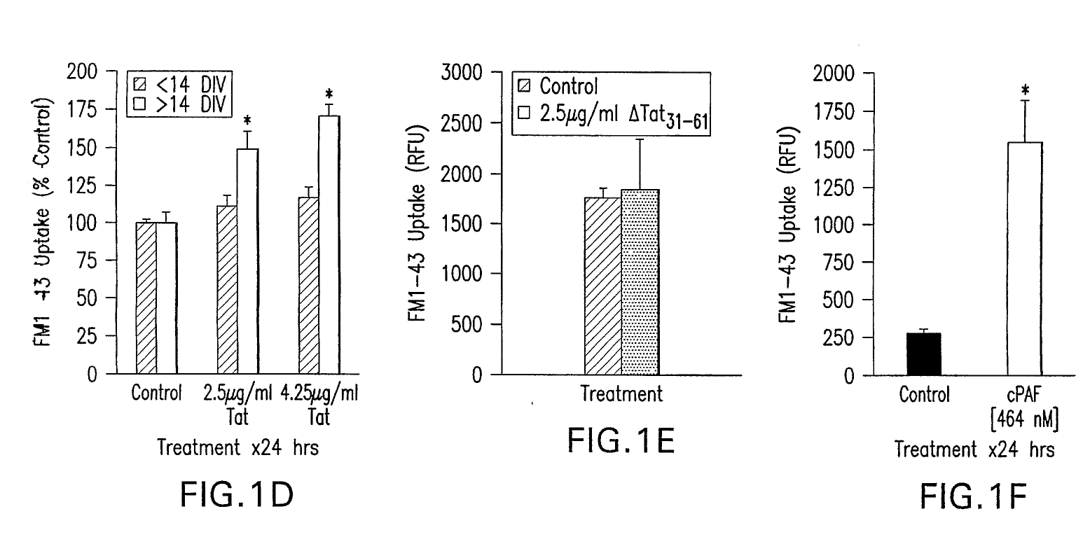

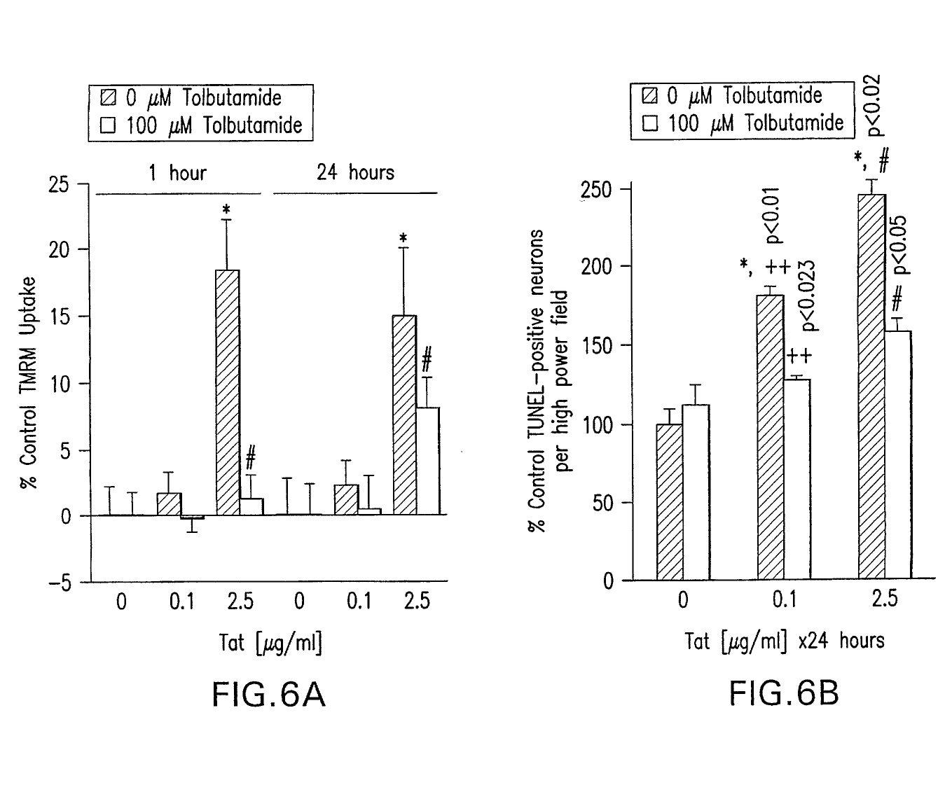

Cortical neuronal cultures were treated with Tat or cPAF for varying periods of time as an in vitro model of pre-synaptic nerve terminal function, using the lipophilic, fluorescent styryl dye N-(3-triethylammoniumpropyl)-4-(4-(dibutylamino)styryl) pyridinium dibromide (FM1-43), which binds to synaptic vesicle membranes and is taken up into nerve terminals during normal activity (i.e., synaptic vesicle recycling). Fluorescent signal can be quantified to number of neurons, and after chemical depolarization with high concentrations of KC1, fluorescent signal is abolished, giving an index of nerve terminal activity in real-time. This model was used to demonstrate that both Tat and PAF increased vesicular uptake (of neurotransmitter) in rodent cortical cultures (FIG. 1). The staining seen with FM1-43 was punctate and largely oriented along neuronal processes (FIGS. 1A, B), and released in a quantal fashion with a depolarizing concentration of KC1, all suggestive of synaptic vesicle recycling (FIG. 1C). Interestingly, Tat caused a dose-dependent increase in FM1-43 uptake that was even more pronounced in aged cultures (FIG. 1D), perhaps due to incomplete maturation of synaptic receptors in the younger cultures [Gelbard, H. A., et al., J Virol, 1994. 68(7): p. 4628-35]. However, an inactive mutant Tat protein had no effect on FM1-43 uptake, indicating the effect was a biologically specific effect of Tat (FIG. 1E). PAF caused an even more robust increase in FM1-43 uptake (FIG. 6F), in keeping with its role as a mediator of pre-synaptic glutamate release [Clark, G. D., et al., Neuron, 1992. 9(6): p. 1211-6].

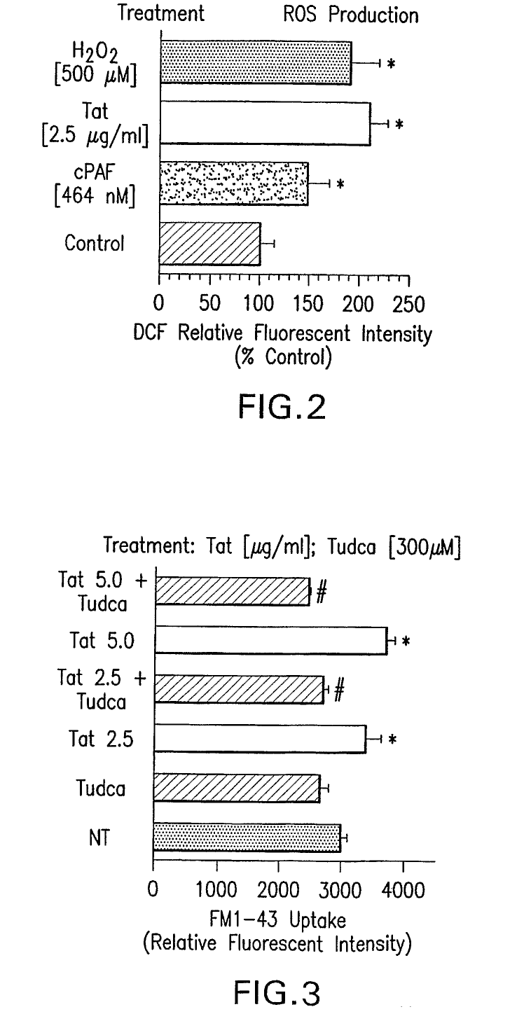

Since reactive oxygen species (ROS) can be a final common mediator of neuronal dysfunction in HAD and other neurodegenerative diseases, and because neuronal synapses and synaptic function can be particularly vulnerable to oxidative stress [Mattson, M. P. and D. Liu, Neuromolecular Med, 2002. 2(2): p. 215-31], ROS production was measured in response to Tat and PAF. Twenty-four hour treatment with either PAF or Tat increased ROS production in primary rodent cortical neurons by approximately 50% and 100% respectively (FIG. 2).

Interestingly, the Tat-induced increase in FM1-43 uptake was completely blocked by the antioxidant bile liver acid antioxidant tauroursodeoxycholic acid (TUDCA) (FIG. 3). These data are in concordance with recent evidence that ROS can directly enhance synaptic transmission [Kamsler, A. and M. Segal, Mol Neurobiol, 2004. 29(2): p. 167-78] [Giniatullin, A. R. and R. A. J Physiol, 2003. 552(Pt 1): p. 283-93] [Chen, B. T., M. V. J Neurophysiol, 2001. 85(6): p. 2468-76]. It is important to point out that enhanced synaptic transmission can come with a cost to the ultimate fate of the neuron.

Changes in mitochondrial function that could be associated with the increased vesicular activity were evaluated. Mitochondria generate the energy for synaptic transmission in the form of ATP, and as a byproduct of this oxidative phosphorylation, they are also one of the primary producers of intracellular ROS. Two key mitochondrial parameters were examined in response to Tat and PAF: 1) mitochondrial membrane potential (Δ_{Ψm}) [as assessed by mitochondrial uptake of the lipophilic cationic dyes tetramethylrhodamine ethyl or methyl ester (TMRE or TMRM respectively)], and 2) ATP production (as measured by luciferase assay). The electronegative mitochondrial membrane potential provides the driving force for calcium buffering and ATP production by the mitochondria, and as such, Δ_{Ψ}m is frequently used as an indicator of mitochondrial health and energetic capacity. Moreover, fluctuations in Δ_{Ψ}m can trigger mitochondrial release of pro-apoptotic factors, an event most frequently associated with a loss of Δ_{Ψm}.

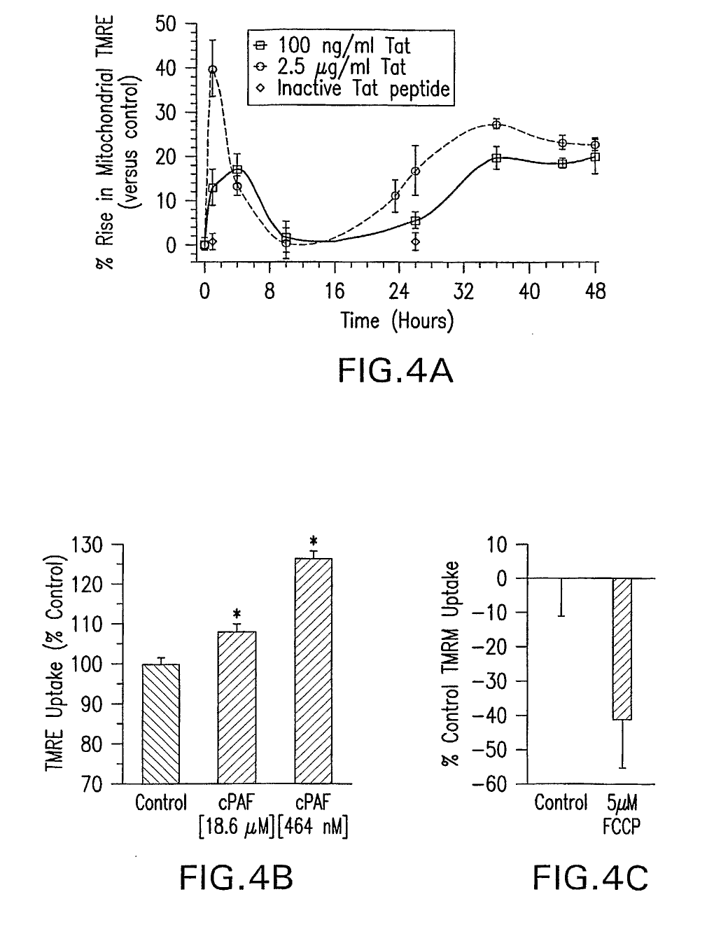

To the contrary, however, Tat treatment of rodent cortical neurons resulted in a dose-dependent, biphasic increase in Δ_{Ψm} over 48 hours (FIG. 4A). A low dose of Tat (100 ng/ml) caused a gradual increase in Δ_{Ψ}ₘ, peaking with a 17% increase in mitochondrial TMRE uptake at 4 hours (p<0.004), then declining to baseline by 14 hours, before rising again (6% increase vs. control vehicle) 26 hours after application, followed by a plateau at 36 hours that persists until the end of the analysis (48 hours) (19% increase vs. control vehicle) (FIG. 4A, squares). In contrast, a higher dose of Tat (2.5 μg/ml), caused a sharp increase in Δ_{Ψm} versus control, peaking at a 39% increase over control by 1 hour (p<0.0003) before declining to control level by 10 hours, then increasing again to a second peak of 27% vs. control at 36 hours, followed by a plateau of 23% vs. control at 44 hours that also persisted until the end of the analysis (48 hours) (FIG. 4A, circles). Incubation of cortical cultures with the biologically inactive Tat mutant (Δ31-61) at either 1 or 26 hours resulted in TMRE uptake values that were indistinguishable from control vehicle (FIG. 4A, diamonds), demonstrating that Tat's effect on mitochondrial hyperpolarization was biologically specific.

In addition, both PAF and TNF-α treatments also caused similar dose dependent increases in Δ_{Ψ}m, suggesting that this effect can be broadly generalizable to HIV neurotoxins in the context of HAD. Notably, the mitochondria-depolarizing protonophore trifluoromethoxycarbonylcyanide phenylhydrazone FCCP (5 μM), substantially diminished TMRE/M signal, thus serving as an additional control that Tat, PAF, and TNF-α were indeed increasing Δ_{Ψ}m.

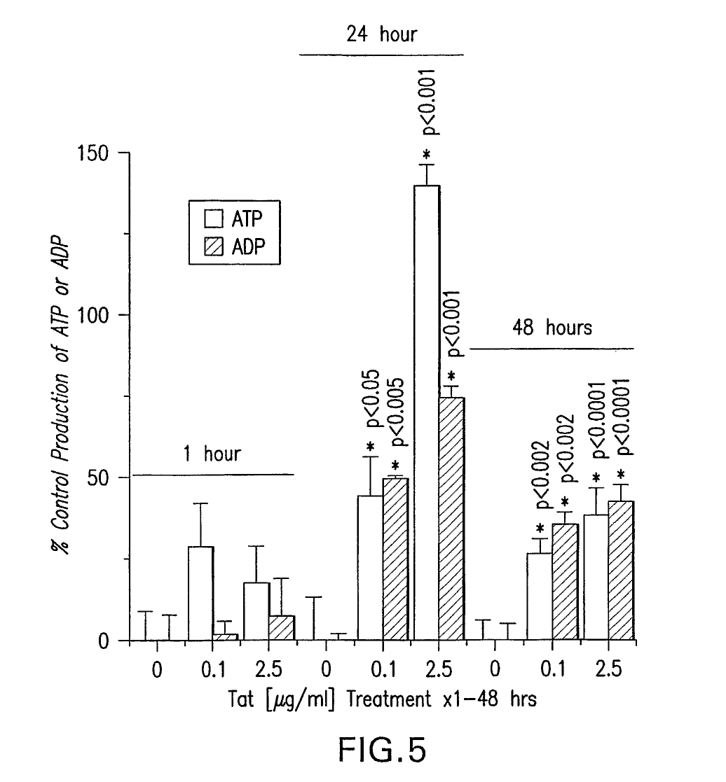

There was also a concomitant, dose-dependent rise in ATP production and ATP/ADP ratios with the Tat induced mitochondrial hyperpolarization, as would be expected from the increased driving force on the mitochondrial F₁F₀-ATPase (FIG. 5). After 1 hour treatment with 0.1 and 2.5 μg/ml Tat, ATP but not ADP levels were slightly elevated versus control, and the ATP/ADP ratio was significantly elevated for the 0.1 μg/ml dose of Tat. After 24 hour treatment, 0.1 and 2.5 μg/ml Tat elevated both ATP and ADP levels, but the ATP/ADP ratio was only significantly increased for the 2.5 μg/ml dose of Tat. By 48 hours, ATP and ADP levels were still elevated for both doses of Tat, but less so, and the ATP/ADP ratio had begun to decline, perhaps indicating that the neurons were entering a stage of ATP deprivation from constant over-activity.

The dose-dependent, Tat-induced rise in mitochondrial membrane potential could, however, be blocked by the potassium ATP (KATP) channel antagonist Tolbutamide (FIG. 6A), which had the further effect of attenuating apoptotic cell death (FIG. 6B). Tolbutamide's principal action is thought to occur via inhibitions of KATP channels [Liss, B. and J. Roeper, Mol Membr Biol, 2001. 18(2): p. 117-27], although it activates glycolysis as well [Kaku, K., Y. Inoue, and T. Kaneko, Diabetes Res Clin Pract, 1995. 28 Suppl: p. S105-8], and has mitochondrial uncoupling properties [Smith, P. A., P. Proks, and A. Moorhouse, Pflugers Arch, 1999. 437(4): p. 577-88].

These data demonstrate that blocking mitochondrial hyperpolarization can have a protective effect on neurons. Thus, this approach represents a therapeutic avenue of tremendous importance. A related therapeutic avenue is the use of protease inhibitors to restore mitochondrial bioenergetics, or more specifically, to block a detrimental rise in mitochondrial polarization.

### Example 2

### Approaches to Normalize Synaptic Transmission in Models of Post-Synaptic Injury

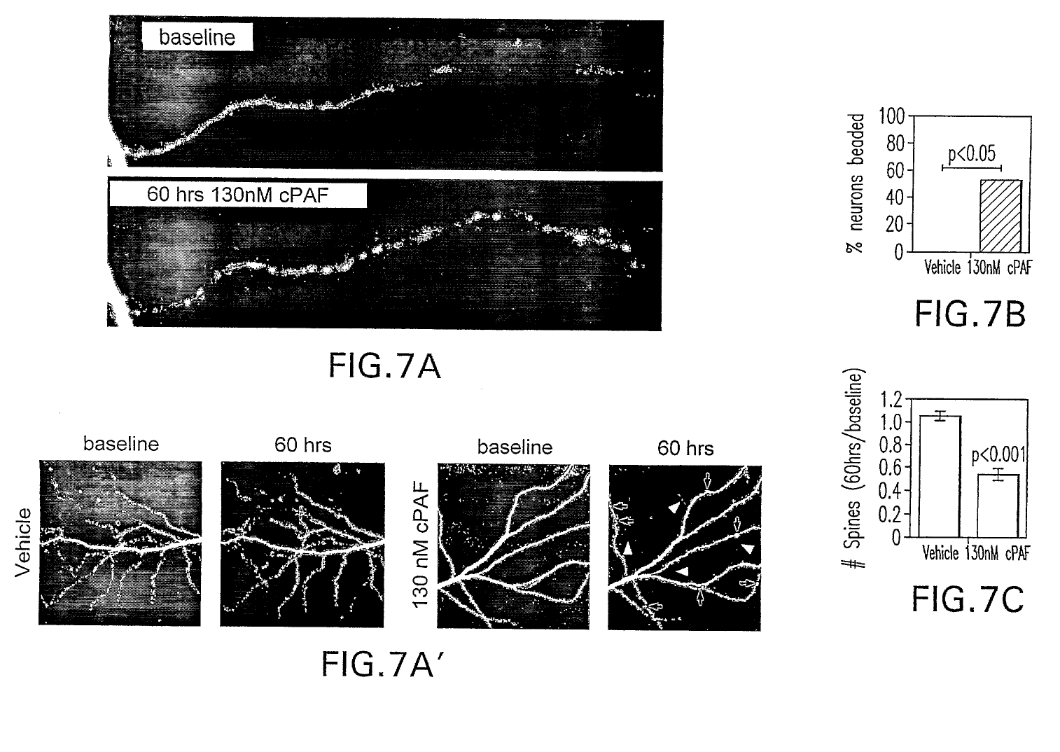

An additional pathologic consequence of exposure to HIV-1 neurotoxins is an increase in oxidized cellular phospholipids that can bind to and activate the PAF-R [Marathe, G. K., et al., Vascul Pharmacol, 2002. 38(4): p. 193-200]. Both energetic stress and PAF-R signaling have the potential to impair post-synaptic neurotransmission. Thus, a morphologic correlate of dendrite damage in HAD was identified, which is activity-induced dendritic swelling or “beading” in the presence of the HIV neurotoxin cPAF. This beading is also accompanied by impairment of synaptic activity. FIG. 7 shows that exposing hippocampal neurons in vitro to sublethal (130 nM) concentrations of cPAF for 60 hours results in loss of dendritic spines (but not neurites) and dendrite beading, without cell death.

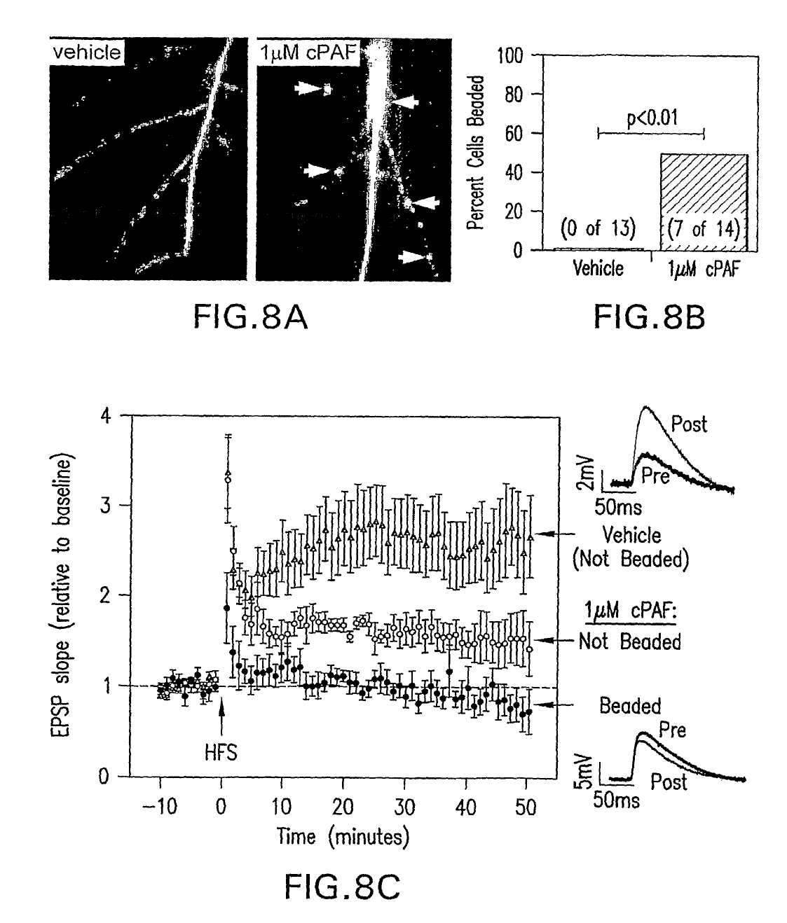

Additionally, in an acute hippocampal slice model, shorter cPAF exposures (1 μM for 30-60 minutes) increased neuronal susceptibility to beading in response to synaptic activity, and results in failure of long-term potentiation in the beaded dendrites (FIG. 8). Increased neuronal susceptibility to beading in response to synaptic activity was also observed in hippocampal cultures following brief (1 hour) cPAF exposures.

Thus, PAF can lower the threshold for synaptic injury, and that by impairing synaptic function, beading can serve as an important functional marker of dendritic injury, and can underlie the reversible impairments of neuronal function seen in HAD. Equally important, the in vitro and in vivo models of dendritic injury disclosed herein are sensitive and reproducible, and have great utility to determine the ability of adjunctive therapies to restore function (i.e., synaptic transmission) during exposure to HIV-1 neurotoxins.

### Example 3

### Bioenergetic Defects in Neurons Exposed to HIV-1 Neurotoxins

HIV neurotoxins can increase vesicle recycling, mitochondrial membrane potential, ATP/ADP ratios, and reactive oxygen species. Furthermore, blocking the Tat-induced rise in Δ_{Ψ}m protects the neurons against apoptotic cell death. Thus, the observed increases in Δ_{Ψ}m are not simply a compensatory response to increased metabolic demand, since in that case blocking the rise in Δ_{Ψ}m would likely lead to energetic failure and exacerbate cell death.

Thus, increased Δ_{Ψ}m in response to HIV neurotoxins could be a pathogenic mechanism leading to excessive production of ROS and ATP, resulting in synaptic stress, excitotoxicity, and eventual demise of synaptic contacts and dendritic arbor, i.e., “synaptic apoptosis” [Mattson, M. P., J. N. Keller, and J. G. Begley, Exp Neurol, 1998. 153(1): p. 35-48]. These pathogenic mechanisms could contribute to the reversible component of HAD, but if left unchecked, could ultimately result in permanent neurologic deficit with or without cell loss.

In order to determine if HIV-neurotoxin-induced mitochondrial hyperpolarization is associated with a rise in NADH/NAD⁺ ratio and increased oxygen consumption by the electron transport chain (ETC) and to further clarify Tat and PAF's effects on oxidative phosphorylation primary rodent cortical or hippocampal neurons are treated with a range of doses of Tat (10 ng/ml, 100 ng/ml, and 2.5 μg/ml) and cPAF (2 nM, 20 nM, and 500 nM) over 1, 4, 8, 12, 24, 36, 48, and 72 hours, and NADH/NAD⁺ ratio and rates of O₂ consumption at these time points assessed. ATP/ADP ratios and Δ_{Ψ}m are also assessed at these time points to verify previous findings. If all components of oxidative phosphorylation are functioning properly, then by increasing the proton driving force through the mitochondrial F₁F₀-ATPase, a mitochondrial hyperpolarization is associated not only with increased ATP production as disclosed herein, but also with increased O₂ consumption and increased NADH/NAD⁺ ratio.

The doses of Tat and PAF described herein have been determined by previous studies to represent sub-threshold (i.e., no measurable effects), sub-lethal (measurable effects on intracellular parameters but induces little cell death at 18-24 hours), and toxic (strong effects on intracellular parameters and induces apparent cell death at 18-24 hours or greater) in neuronal culture systems. These doses have been selected to cover the range of responses expected for the cellular functions being assessed. However, for any given assay, a full dose-response calibration is performed, and Tat or cPAF doses altered, if warranted. Additionally, specificity of effects is confirmed by substituting Tat with an equimolar amount of a biologically inactive mutant Tat protein (ΔTat3 1-61) [Gurwell, J. A., et al., Neuroscience, 2001. 102(3): p. 555-63] or applying cPAF with the PAF receptor antagonists WEB 2086 (10 μM) or BN52021 (10 μM).

In order to determine if the ETC or the mitochondrial F₁F₀-ATPase are responsible for the rise in Δ_{Ψm}) if blocking the hyperpolarization results in return of ATP/ADP ratios to baseline levels, and to determine if the F₁F₀-ATPase or the ETC is responsible for the Tat or PAF-induced rise in Δ_{Ψm}, primary cortical neuron cultures are treated with Tat or PAF at the doses and time points above, in the presence of the F₁F₀-ATPase inhibitor oligomycin (5 μg/ml, strong depolarizing dose) [Ward, M. W., et al., J Neurosci, 2000. 20(19): p. 7208-19], and the effects on mitochondrial membrane potential and ATP/ADP ratios are monitored.

If oligomycin partially or fully blocks the Tat or PAF-induced rise in Δ_{Ψm}, then reversal of the F₁F₀-ATPase is at least in part responsible for the mitochondrial hyperpolarization. In this case, Tat or PAF+oligomycin induces a rise in ATP/ADP ratios versus Tat or PAF alone, due to reduced consumption of ATP by the reversed F₁F₀-ATPase.

If the F₁F₀-ATPase is functioning normally—i.e. passing protons into the mitochondria and generating ATP—then co-incubation with oligomycin augments the hyperpolarizing effects of Tat and PAF, and reduce ATP/ADP ratios, thus suggesting that the ETC, and not the F₁F₀-ATPase, is responsible for a hyperpolarized Δ_{Ψm} measured after Tat or PAF exposure.

In order to determine if the rise in Δ_{Ψm} is accompanied by increased ROS production, Cytochrome-C (CytC) release by the ETC, and caspase activation, and to determine if increased ROS production from the ETC is a consequence of the Tat or PAF-induced rise in Δ_{Ψm}, primary cortical neuron cultures are treated with Tat (10 ng/ml, 100 ng/ml, and 2.5 μg/ml) and PAF (2 nm, 20 nm, and 500 nm) over 1, 4, 8, 12, 24, 36, 48, and 72 hours, with or without the complex I inhibitors diphenyleneiodonium and rotenone or the complex III inhibitors antimycin and myxothiazole.

Mitochondrial complexes I and III are the chief ROS producers of the ETC [Nicholls, D. G., Int J Biochem Cell Biol, 2002. 34(11): p. 1372-81]. ROS levels are assessed by with the oxidizable dye indicator 5-(and-6)-chloromethyl-2′,7′-dichlorodihydrofluorescein diacetate, acetyl ester (CMH₂DCFDA, abbreviated as “DCF”) (Molecular Probes, Eugene, Oreg.) as described herein. If ROS levels are higher in Tat/PAF treated cultures, and this effect is blocked by rotenone and/or myxothiazole, then the hyperpolarization is contributing to enhanced ROS production from the ETC in Tat/PAF treated neurons.

To determine if Tat/PAF-induced mitochondrial hyperpolarization results in increased CytC release from mitochondria, rodent cortical neurons are treated with Tat or PAF as above, then assayed for release of cytochrome c using a commercially available kit (http://www.emdbiosciences.com/product/QIA87). If increased release of CytC from mitochondria is observed, it can be confirmed that mitochondrial hyperpolarization is responsible for this effect by blocking the hyperpolarization with low-dose FCCP (100 nM) and the ETC complex inhibitors listed above, as well as tolbutamide and any other hyperpolarization-inhibiting compounds identified herein.

Complex II and IV inhibitors, succinate-q reductase (TTFA) and potassium cyanide (KCN) respectively can also be used herein. Complexes II and IV contribute to ROS production, but generally not as much as complexes I and III [Nicholls, D. G., Int J Biochem Cell Biol, 2002. 34(11): p. 1372-81]. Further, in addition to Caspase 9, activation of Apaf1, another key component of the CytC/Apaf1/Caspase9 apoptosome, and Caspase 3 are also examined.

Further, Tat or PAF±glucose or NADH are co-incubated, and Δ_{Ψm}, O₂ consumption, NADH levels, ATP/ADP ratios, and apoptotic cell death assessed at the 24, 48, and 72 hour time points. NADH and glucose are titrated to select doses that are not toxic to the neuronal cultures.

To assess the possibility that the decreased ATP production that results from blocking the Tat-induced rise in Δ_{Ψm} simply shifts the cell death continuum from apoptosis to necrosis, dual labels for apoptosis and necrosis are used as previously described [Perry, S. W., L. G. Epstein, and H. A. Gelbard, Biotechniques, 1997. 22(6): p. 1102-6].

KATP channel antagonists: As disclosed herein, the KATP channel antagonist and hypoglycemic agent Tolbutamide block the rise in Δ_{Ψm} from the HIV-1 virotoxin Tat and protect against cell death. Tolbutamide's principal action is thought to occur via inhibitions of KATP channels [Liss, B. and J. Roeper, Mol Membr Biol, 2001. 18(2): p. 117-27], although it activates glycolysis as well [Kaku, K., Y. Inoue, and T. Kaneko, Diabetes Res Clin Pract, 1995. 28 Suppl: p. S105-8], and has mitochondrial uncoupling properties [Smith, P. A., P. Proks, and A. Moorhouse, Pflugers Arch, 1999. 437(4): p. 577-88]. The first and third mechanisms are the most likely candidates for tolbutamide's protective and stabilizing effects based on the data disclosed herein, as enhancing glycolysis would only further increase Δ_{Ψm}, ATP, and ROS production. Controlled uncoupling, or regulation of mitochondrial permeability to H+ or other ions could underlie protective actions of several drugs disclosed herein, notably those that link pro-apoptotic mitochondrial proteins with mitochondrial membrane permeability (see rationale for “protease and enzyme inhibitors” below). In addition, aberrant control of mitochondrial polarity represents a link between mitochondrial status and dendrite beading, via volume-regulated anion channels (VRACs).

However, because there is concern with reports of cardiac toxicity with Tolbutamide [Meier, J. J., et al., Heart, 2004. 90(1): p. 9-12] other KATP antagonists with known neuronal action, but exhibiting lower (e.g. glibenclamide aka glyburide) [Riveline, J. P., et al., Diabetes Metab, 2003. 29(3): p. 207-22] or no (e.g. meglitinide analogs) [Schwanstecher, C. and D. Bassen, Br J Pharmacol, 1997. 121(2): p. 193-8] cardiac toxicity are expected. The mitochondrial KATP channel specific antagonist 5-hydroxydecanoic acid (5-HD), and the KATP channel agonist diazoxide, are used as controls to further dissect KATP antagonist protective mechanisms.

Antioxidant compounds: As disclosed herein, Tat and PAF induce ROS production in neurons, which could stem from increased oxidative phosphorylation and ATP production. Mitochondrial production of ROS leads to enhanced excitotoxic synaptic activity, leading to synaptic damage, or activates cell death pathways directly. Synapses are particularly vulnerable to oxidative stress. As disclosed herein, the antioxidant compound Tauroursodeoxycholic acid (TUDCA) protects against Tat-induced increases in synaptic activity. Together these data indicate that antioxidant compounds protect against synaptic damage and dysfunction in HAD.

Candidate antioxidants are evaluated with the herein disclosed biological models of HAD, including the mitochondria-targeted antioxidants Mito-Coenzyme Q10 (Mito-CoQ) and Mito-VitaminE (Mito-E) [Green, K., M. D. Brand, and M. P. Murphy, Diabetes, 2004, 53 Suppl 1: p. S110-8]—as well as regular antioxidants such as Coenzyme Q10 and ibedenone [Smith, R. A., et al., Proc Natl Acad Sci U S A, 2003. 100(9): p. 5407-12] [Green, K., M. D. Brand, and M. P. Murphy, Diabetes, 2004. 53 Suppl 1: p. S110-8].

Candidate compounds are tested for their efficacy in preventing pathologic outcomes against Tat and PAF treatment in primary assay systems that provide physiological and morphologic correlates of neuronal dysfunction HAD, i.e. whether they block mitochondrial hyperpolarization and cell death, prevent aberrant synaptic transmission and dendrite loss, and/or prevent dendrite swelling or “beading”.

Using the Tat and PAF doses and a time course disclosed herein, the compounds identified herein are tested for their ability to block Tat and PAF-induced mitochondrial hyperpolarization, as assessed by the TMRM assay (below), and cell death, as assessed by a combined assay for both necrosis and apoptosis [Perry, S. W., L. G. Epstein, and H. A. Gelbard, Biotechniques, 1997. 22(6): p. 1102-6]. All potential therapeutic compounds are first be titrated in full dose response curves to determine non-toxic doses for neurons. Dose ranges of Tat and PAF are extended if necessary to clearly determine whether protective effects exist.

Understanding potential interactions between HAART (particularly protease inhibitors [PIs] and nucleoside reverse transcriptase inhibitors [NRTIs]) and adjunctive neuroprotective agents disclosed herein is of great value in terms of defining future regimens of HAART that are tailored specifically to the amelioration of CNS disease and/or use in combination with specific adjunctive regimens that target mitochondrial bioenergetics, synaptic dysfunction, and neuronal apoptosis. For example, pro- and anti-apoptotic proteins could participate not only in pathologic mechanisms at the synapse, but also physiologic processes of synaptic regulation, such as growth cone rearrangement or synaptic strengthening [Jonas, E., J Bioenerg Biomembr, 2004. 36(4): p. 357-61]. Thus PIs that alter the processing of mitochondrial apoptotic regulatory proteins such as BCL-xL could both impact mitochondrial and cellular/synaptic function—as well as cell fate.

Thus, the effect of PIs (e.g. Ritonavir) and NRTIs (e.g. Tenofovir) (alone and in combination), as well as standard PI-using (kaletra [lopinavir/ritonavir]+efavirenz) and PI-sparing (efavirenz+AZT+3TC) HAART regimens, are evaluated on the protective and functional outcomes of neuronal exposure to promising adjunctive neuroprotective agents described herein. Outcome measures include normalization of mitochondrial bioenergetics and synaptic vesicle recycling after Tat or PAF exposure. These assays reveal whether potential mitochondrial toxicity by NRTIs, or mitochondrial protection by PIs [Matarrese, P., et al., J Immunol, 2003. 170(12): p. 6006-15], modulates the effect of candidate therapies. These assays also determine whether a CNS-penetrating PI such as Indinavir exerts effects on mitochondrial hyperpolarization in neurons in the same way that some PIs have been shown to exert such effects on T cells [Matarrese, P., et al., J Immunol, 2003. 170(12): p. 6006-15].

TABLE 1
Antiretrovirals

| Drug Category | Specific compound |
|---|---|
| PI | Indinavir (penetrates the CNS); Lopinavir, |
| | Ritonavir, Atazanavir |
| NRTI | AZT, 3TC, Tenofovir |
| NNRTI | Efavirenz |
| HAART | 1) Atazanavir + Tenofovir + Ritonavir |
| | 2) AZT + 3TC + Efavirenz |
| | 3) Lopinavir + Ritonavir + Efavirenz |
| | (HAART regimens) |

Adjunctive neuroprotective agents provided herein (at doses known to be effective in the in vitro assay systems) are combined with different antiretrovirals, starting with individual drugs alone (PIs, NRTIs) and then progressing to the three defined HAART regimes (see Table X, above). Selection of dose ranges is based on physiologically achievable drug levels and/or the EC50 and EC99 for each drug. Thus, as an example, Atazanvir is used at doses between 1-100 nM [Colonno, R. J., et al., Antimicrob Agents Chemother, 2003. 47(4): p. 1324-33] based on it's ED50 dose (2-8 nM) for inhibiting viral replication in HIV-1 isolates resistant to other protease inhibitors.

The effects of other adjunctive neuroprotective agents that do not directly affect mitochondrial bioenergetics (e.g., compounds disclosed herein) are evaluated for their effects on mitochondrial membrane potential and synaptic activity in neurons exposed to Tat or PAF.

Reagents: Carbamyl-PAF (c-PAF; abbreviated “PAF” herein), a non-hydrolyzable form of PAF, is obtained from Biomol (Plymouth Landing, Pa.).

Cell Culture: Cells that are used in the compositions and methods disclosed herein include:

(1) Primary rat cerebellar granule neurons (CGNs). CGNs represent a highly homogenous population of primary neurons which are susceptible to Tat and PAF-mediated cell killing and have been used in many of previous studies. CGN cells are isolated according to published procedures [Maggirwar, S. B., et al. 1999. J Neurochem 73:578-86] [Tong, N., et al. 2001. Eur J Neurosci 13:1913-22].

(2) Primary rat cortical neurons (CN). CN are readily established, and available in significant cell numbers; they have the particular advantage that they fully reprise all the neuronal circuitry necessary to drive striatal projection neurons and are therefore used to identify electrophysiologic parameters of synaptic transmission to develop a functional bioassay for measuring the neuroprotective efficacy of potential therapeutic agents. Like CGN, CN are susceptible to Tat and PAF-mediated cell killing as well as Tat and PAF-mediated mitochondrial dysfunction and synaptic apoptosis. These cells are isolated and maintained using methods described herein and in [Perry, S. W., et al. 2004. J Neurosci Res], incorporated by reference herein for its teaching of these methods.

(3) Primary rat hippocampal neurons (HN). The hippocampus plays a central role in learning and memory—processes which are adversely affected in HIV-associated neurologic disease. Furthermore, HN are susceptible to Tat and PAF-mediated cell killing [Kruman, 11, et al. 1998. Exp Neurol 154:276-88] [Nath, A., et al. 1996. J Virol 70:1475-80] as well as Tat-mediated excitotoxicity [Song, L., et al 2003. J Neurovirol 9:399-403]. These cells also undergo reductions in dendritic arborization in response to Tat [Maragos, W. F., et al. 2002. J Neurochem 83:955-63] or secretory products from HIV-1 infected monocyte-derived macrophages [Zheng, J., et al. 2001. Neurotox Res 3:443-59]. HNs are be prepared from embryonic day 18 rats by modification of the protocol by Brewer [Brewer, G. J., et al. 1993. J Neurosci Res 35:567-76]. In brief, hippocampi are dissected from a litter of E18 embryonic rats, dissected free of meninges and other tissue, and incubated in 2.0 ml of Ca⁺/Mg⁺-free Hanks balanced salt solution (HBSS) (with 10 mM HEPES, pH7.3) with PSN antibiotics (penicillin 50 mg/L; streptomycin 50 mg/L; neomycin 100 mg/L) plus 0.5 ml for 2.5% trypsin (for 0.25% final) for 15 min at 37° C. per brain. After the 15 minute incubation, trypsin is removed, cells are washed twice with HBSS (with Ca⁺/Mg⁺), then dissociated in growth media (below) by Pasteur pipette trituration by 8-10 passages through a 0.9 mm bore 1000 μl blue pipette tip. Dissociated cells are counted by trypan blue viability assay and plated in cell culture plates at 0.5-0.6×10⁵ cells/cm², on poly-D-lysine-coated cell culture plastic or sterile glass coverslips. The plating and maintenance media consists of Neurobasal with B27 supplement (trademark Life Technologies, Gaithersburg, Md.) as described by Brewer [Brewer, G. J., et al. 1993. J Neurosci Res 35:567-76]. This media formulation inhibits the outgrowth of glia resulting in a neuronal population that is 98% pure; thus glial inhibitors are unnecessary. Cells are cultured for 10-21 days at 37° C. in a humidified atmosphere of 5% CO_{2/95}% air, changing media every 4 days; cells are used for experiments at days in vitro (DIV) 14-21.

(4) Mixed glial-neuronal cells. Primary human fetal neurons are obtained from ScienCell, and are used to confirm key results, since they are susceptible to Tat and PAF mediated cell killing, and killing by monocyte-conditioned medium [Gelbard, H. A., et al. 1994. J Virol 68:4628-35] [New, D. R., et al. 1997. J Neurovirol 3:168-73]. Primary human fetal neurons lack the regional homogeneity that characterizes primary rat neurons, and cannot be obtained in pure culture without contaminating astrocytes (20-25% of the cell population, typically) and microglia (˜5% of the cell population, typically). These properties make primary human fetal neurons undesirable when one wishes to examine highly homogenous neuronal populations. However, the presence of glia within the cultures, combined with their human origin, offers advantages in terms of providing a confirmatory model system that more closely approximates the human condition in vivo.

(5) Primary microglia, of both rat and human origin. Primary rat microglia are isolated using previously described methods [Patrizio, M., et al. 2001. J Neurochem 77:399-407]. These cells are used for all initial screening of candidate therapeutic molecules for the potential to influence microglia activation. It should be noted that rat microglia isolated by this method are efficiently activated by HIV-1 Tat [Patrizio, M., et al. 2001. J Neurochem 77:399-407]. Human fetal microglia are used in confirmatory experiments, to verify key findings. These cells are obtained from ScienCell, which provides highly purified (>90% CD11b+) cultures. Mixed primary rat glial cultures are established from 1-day postnatal Wistar rat cerebral cortex and grown in Basal Eagle's Medium supplemented with 10% FBS plus glutamine and antibiotics (penicillin/streptomycin). The cells are plated on poly-L-lysine-coated plastic at a density of 2.5×10⁴ cells/cm₂ in 24-well culture plates. Microglial cells re removed from 12 to 15-day primary glial cultures by mild shaking and plated on uncoated plastic at a density of 1.5×10⁵ cells/cm2 in 48-well culture plates. After 20 min, the cultures are washed with fresh medium to remove non-adherent cells and grown for 2 to 3 days. These cultures are typically 99% pure microglia/macrophages, as assessed by staining for the macrophage marker ED1. Added note:

(6) Primary monocytes. Immunomagnetic isolation of CD14⁺ human monocytes [Wang, X., et al. 2003. J Virol 77:7182-92] is used to isolate monocytes from peripheral blood mononuclear cells immediately following phlebotomy. Purity of the isolated cell population is verified by flow cytometric analysis using a CD 14-specific monoclonal antibody; cells are used if >95% CD14⁺. Positive immunomagnetic selection is used for CD14+ human monocytes (Miltenyi-Biotec). However, since positive selection results in cellular activation, monocyte populations are isolated from a panel of normal donors by both positive selection (using CD14-conjugated magnetic beads) and negative selection (i.e., “untouched” cells). Results are compared for both populations, as well as purity (as assessed by immunophenotyping using CD14, CD68 and MHC class II).

Monitoring alterations in Δ_{Ψm} with TMRM: In the relevant methods described herein, changes in mitochondrial membrane potential (Δ_{Ψm}) in response to Tat or PAF treatment is assessed by TMRM, which must equilibrate across the plasma membrane before entering the mitochondria; and in whole cell applications dye concentration in the mitochondria is dependent on both plasma membrane potential (Δ_{Ψp}) and A_{Ψm}. At sufficiently low dye concentrations (TMRM =1 nM, i.e. high cell/dye ratio) and normal membrane potentials, ≧95% of the dye is in the mitochondria, and ≦1% in the media, which results in an approximately 100× sensitivity of the dye to Δ_{Ψm} over Δ_{Ψp}, given equivalent changes in plasma or mitochondrial membrane potential [Rottenberg, H. and S. Wu, Biochim Biophys Acta, 1998. 1404(3): p. 393-40].

For these methods, primary rodent neurons are treated with reagent under normal culture conditions for the indicated time period, followed by removal of the media, 1×2 minute wash in pre-warmed 37° C. HBSS plus 10 mM glucose and 10 mM HEPES (HBSS+), then incubation in HBSS+ with 1 nM TMRE/M. Following equilibration of the dye for 20 minutes to ensure distribution across the mitochondrial membrane, the cells are imaged while remaining in the 1 nM TMRE/M solution, as is necessary for a continued dye equilibrium state. Random field images are taken using an Olympus IX-70 microscope and 40× objective (fluorescent excitation: 545; emission: 610) and Apogee KX32ME CCD camera, and the mean relative fluorescent unit (RFU) value of the neuronal soma and processes (excluding mitochondria deficient regions) is quantified using Scanalytics IPLab software.

Measuring production of intracellular ROS: Generation of ROS in response to Tat and PAF is assessed with the oxidizable dye indicator 5-(and-6)-chloromethyl-2′, 7′-dichlorodihydrofluorescein diacetate, acetyl ester (CM—H₂DCFDA, abbreviated as “DCF”) (Molecular Probes, Eugene, Oreg.). Upon entering the cell by passive diffusion, the acetate groups are cleaved by intracellular esterases, and the thiol-reactive chloromethyl group binds to intracellular thiols, rendering the CM—H₂DCF product trapped within the cell. Oxidation by ROS including hydrogen peroxide (H₂O₂), hydroxyl radical (HO⁻), peroxyl radical (HOO⁻), or peroxynitrite anion (ONOO⁻) [Carter, W. O., P. K. Narayanan, and J. P. Robinson, J Leukoc Biol, 1994. 55(2): p. 253-8] [manufacturer's data] leaves the final fluorescent product CM-DCF, which is then imaged (excitation: 485, emission: 538) and quantitated in the same fashion as TMRM (above) as an indicator of relative levels of ROS in the culture.

ATP and ADP Measurement: Adenosine di- and tri-phosphate (ADP and ATP respectively) levels in cortical cultures were measured after treatment using a kit from Cambrex (Rockland, Me.). Briefly, cortical neurons were plated in white poly-d-lysine coated 96 well plates at a density of 32,000 cells/well. Each treatment group contained five replicate wells. After treatment, ATP and ADP levels were then measured using an ATP/ADP assay kit (Cambrex, Rockland, Me.). This kit assays ATP levels via the luciferase reaction, then assays ADP levels by a proprietary method of ADP to ATP conversion; the difference in luminescence signal pre-and post-ADP conversion represents the ADP levels in the culture. Luminescent signal was integrated over 1 second and read on a Packard LumiCount microplate luminometer (Meriden, Conn.). For each condition, data represents the mean±SEM ATP or ADP signal of five replicates per condition, expressed as percent increase over corresponding untreated, time-matched control. Background luminescence was negligible, but nonetheless was subtracted from all readings before data analysis. Experimental conditions were run two or more times with similar results.

FM1-43 uptake assay: Total spontaneous activity dependent vesicular uptake was assessed using the lipophilic styryl dye N-(3-triethylammoniumpropyl)-4-(4-(dibutylamino)styryl) pyridinium dibromide (FM1-43). FM1-43 is an amphipathic molecule with a +2 charge that prevents it from passively crossing membranes [Ryan, L. A., et al., Cell Mol Biol (Noisy-le-grand), 2002. 48(2): p. 137-50]. It is non-fluorescent in aqueous solution, but becomes fluorescent upon reversibly binding to exposed membrane by partitioning into the lipid bilayer. Therefore extracellular membranes bind FM1-43, which then becomes encapsulated into a vesicle during endocytosis, generating a fluorescent signal from the endocytosed vesicle. Upon subsequent exocytosis, the vesicle fuses with the membrane, releasing its contents, and the FM1-43 dissociates back into the extracellular solution and loses its fluorescence. These features of this dye have made it a useful tool for studying endocytosis and exocytosis, notably synaptic vesicle recycling and size estimation of various synaptic vesicle pools. FM1-43 was loaded in the absence of a depolarizing stimulus to neurons. This method loads FM1-43 into vesicles undergoing both spontaneous miniature synaptic release and spontaneous action-potential (AP) dependent release.

FM1-43 was loaded for 15 minutes, a length of time that has been determined to label spontaneous activity without saturating the vesicle pool [Prange, O. and T. H. Murphy, J Neurosci, 1999. 19(15): p. 6427-38]. Following washout of excess dye, release of the spontaneously endocytosed FM1-43 in a 100 mM KC1 depolarizing bath confirmed the activity dependent nature of the staining [Pyle, J. L., et al., Neuron, 1999. 24(4): p. 803-8], and ensured that all FM1-43 vesicular uptake was released.

Immediately after addition of the depolarizing solution, cells were transferred to a Bio-rad fluoromark plate reader, and release of FM1-43 was monitored at 20 sec intervals using 438 excitation and 605 emission filters. Since background non-vesicular staining by FM1-43 is not released by KC1 [Pyle, J. L., et al., Neuron, 1999. 24(4): p. 803-8], then for each condition, the total loss of FM1-43 signal during the release period equals the total spontaneous activity dependent vesicular uptake of FM1-43 in each well over the loading period. This absolute FM1-43 uptake value reflects the total activity of the culture, and is equivalent to the synaptic or vesicular release probability of the culture [Prange, O. and T. H. Murphy, J Neurosci, 1999. 19(15): p. 6427-38].

Quantifying Cell Death: Cell death is assessed by visualizing fragmented DNA per the TUNEL method as described previously as well [Perry, S. W., et al., J Neurosci Res, 2004. 78(4): p. 485-92], and where necessary, a dual stain with Trypan blue (to identify necrotic cells)/ApopTag reagent [Perry, S. W., L. G. Epstein, and H. A. Gelbard, Biotechniques, 1997. 22(6): p. 1102-6]. Briefly, after treatment in 24 well plates, cells are fixed with Histochoice MB Tissue Fixative (Amresco) then TUNEL-labeled with the ApopTag kit (Chemicon) according to kit instructions. Cells are visualized under Hoffman modulation contrast optics using a 40× objective, and images taken of ten random fields per well from 3 replicate wells per condition. Data is expressed as percent Apoptag-positive neurons [(# Apoptag positive neurons/)(Total neurons))33 100] (or Trypan blue positive neurons where applicable) per field, then field values averaged for a mean cell death value per well. Mean values from 3 or more (see “power” calculations below) wells are averaged for a final mean cell death value for each condition±SEM; the analyses is made without knowledge of the treatment group.

### Example 4

### Approaches To Normalize Synaptic Transmission In In Vitro and Ex Vivo Morphologic Correlates of Post-Synaptic Injury

Severity of HIV dementia correlates better with loss of dendritic architecture than with frank neuronal loss [Everall, I. P., J Neurovirol, 2000. 6 Suppl 1: p. S103-5]. Therefore, while assessing cell death remains an important method by which to better understand and dissect the bioenergetic consequences of Tat and PAF-treated neurons and assess adjunctive neuroprotective agents' therapeutic potential, other biologic outcome markers are needed to better model the reversible metabolic component of HAD. Therefore, the herein disclosed adjunctive neuroprotective agents are evaluated for the ability to reverse or ameliorate synaptic dysfunction in the herein described models of HIV-1 associated neurologic disease.

HIV neurotoxins including Tat and PAF induce a reversible synaptic dysfunction that is morphologically characterized by dendrite beading, and eventually lead to permanent synaptic deficit (i.e. synaptic apoptosis) if the local concentration of HIV-1 neurotoxins increases to irreversibly toxic levels. Tat and PAF increase pre-synaptic terminal activity and induce mitochondrial hyperpolarization, contemporaneously with increased ROS and ATP production. ROS and ATP both induce synaptic activity directly [Cheng, J., et al., Neuroscience, 1998. 82(1): p. 97-106] [Kamsler, A. and M. Segal, Mol Neurobiol, 2004. 29(2): p. 167-78] [Giniatullin, A. R. and R. A. J Physiol, 2003. 552(Pt 1): p. 283-93], and excessive ROS levels lead to synaptic damage. Furthermore, excessive synaptic activity leads to disruption of mitochondrial structure and function via Δ_{Ψ}m-dependent calcium uptake [Rintoul, G. L., et al., J Neurosci, 2003. 23(21): p. 7881-8], which could be augmented by mitochondrial hyperpolarization. Together, these mechanisms could result in a vicious cycle of synaptic disruption and damage in the context of HAD.

Explant hippocampal slices are exposed to HIV-1 neurotoxins±agents that reverse mitochondrial hyperpolarization. Beading and LTP are then be measured:

TABLE 2

| HIV-1 | | | Beading | LTP |
|---|---|---|---|---|
| neurotoxin | Treatment | Δψm | Prediction | Prediction |
| Tat | FCCP | Normalized | Prevented | Achieved |
| | (100 nM) | | | |
| Tat | Vehicle | Hyperpolarized | Occurs | Failure |
| cPAF | FCCP | Normalized | Prevented | Achieved |
| | (100 nM) | | | |
| cPAF | Vehicle | Hyperpolarized | Occurs | Failure |
| Vehicle | FCCP | Slightly | N/A | ? |
| | | Depolarized | | |
| Vehicle | Vehicle | No change | None | Achieved |

As described above, 100 mn FCCP normalizes Δ_{Ψ}m back to baseline levels but not below; here it is used simply to determine the effects of normalizing Δ_{Ψ}m on beading and LTP in this model; it is not being considered as an adjunctive therapeutic agent. Other candidate therapeutics that normalize Δ_{Ψm} (e.g. glibenclamide) are tested in place of FCCP, with a view to use in future animal or human studies.

Hippocampal slice preparation: Brains from anesthetized P14-28 rodents are rapidly removed and cooled in sucrose based artificial cerebrospinal fluid (ACSF) containing in mM: 110 sucrose, 60 NaCl, 3 KCl, 1.25 NaH₂PO₄, 28 NaHCO₃, 10 D-glucose, 0.5 CaCl₂, 7 MgSO₄, 0.6 ascorbate. The brains are blocked and fixed to a specimen stage with cyanoacrylate. Coronal slices (250-400 μm in thickness) are cut with a vibroslicer (World Precision Instruments, Fla., USA) equipped with a Peltier cooling system. The middle four to six slices of each hippocampus (with the entorhinal cortex removed) are placed into a holding chamber prior to use.

Measuring dendritic beading and postsynaptic potentials in single CA1 cells in hippocampal slices: Dendritic arbors of individual CA1 pyramidal cells are imaged while simultaneously recording excitatory postsynaptic potentials in the same cell by patch clamp recordings in whole-cell configuration. Recording pipettes (3-6 Ω resistance) are pulled from 1.5 mm borosilicate glass using a horizontal Flaming/Brown micropipette puller (Sutter Instrument Co, Calif.), fire-polished, and filled with intracellular recording solution (in mM: KCl 20, potassium gluconate 130, EGTA 0.5, HEPES 10, MgSO₄ 2, ATP 2.5, GTP 0.5, pH 7.3). In order to visualize dendrites of the recorded cell, Alexa 568 hydrazide (30 μM) is included in the recording pipette and is injected by small negative current pulses for 15-20 min prior to recording in order to fill the dendritic arbor. Hippocampal slices are bathed in artificial CSF (containing, in mM: NaCl 125, KCl 2.5, NaH₂PO₄ 1.25, NaHCO₃ 25, CaCl₂ 2, MgCl2 1, D-glucose 25, oxygenated and buffered with 5% CO₂). Membrane potentials of CA1 pyramidal cells are recorded in current clamp mode, and postsynaptic potentials are evoked by test pulses of constant-current stimulation applied via a bipolar stimulating electrode placed 50-200 μm away in the stratum radiatum. Bicuculline (10 μM) is included in the bath to isolate excitatory postsynaptic potentials. After at least 10 min of consistent baseline recording, a high-frequency LTP induction stimulus (HFS, consisting of four 1 s, 100 Hz trains delivered every 15 s) is applied. EPSPs are recorded for 60 min following HFS. At the same time, dendrites are imaged at 40× magnification by fluorescence microscopy, using a shutter to limit fluorescent light exposure and prevent phototoxic injury to the slice. Serial images of dendrites are compared to detect development of focal swellings along dendritic shafts following HFS. Cells are scored as beaded if swellings develop along any of their dendrites.

Field potential recording and induction of LTP in hippocampal slices: Single-cell recordings are complemented by extracellular field potential recordings: while single cell recordings allow correlation of electrophysiologic data with dendritic beading in the same cell, extracellular recordings sample responses from populations of dendrites and are less invasive. Extracellular recording electrodes, filled with 2 M NaCl and 2% pontamine blue (electrode impedance 2-4 MΩ), are placed within the stratum radiatum layer of area C1. Field excitatory post-synaptic potentials (EPSPs) are evoked by stimulation of the Schaffer collateral-commissural afferents once every 30 sec and the initial (1-2 ms) slope is measured. Baseline responses are recorded for at least 20 min prior to induction of LTP by tetanic stimulation (four individual 100 Hz trains delivered for 1 sec each at the test intensity with inter-train intervals of 15 sec). Field responses are measured for 1 hr after applying tetanic stimulation; % baseline values are determined from the final 10 min interval recorded. Field potentials are recorded using an Axoclamp-2B amplifier (Axon Instruments, Calif.) and amplified further by an EX1 differential amplifier (Dagan Corporation, Minn., USA). Data acquisition and analyses are performed using pClamp 8 software (Axon Instruments, Calif., USA) on a Pentium IV PC computer (Dell, Ind., USA).

### Example 5

### Adenosine 2A (A_{2A}) Receptor Antagonist Protects Against Tat-Induced Neuronal Apoptosis

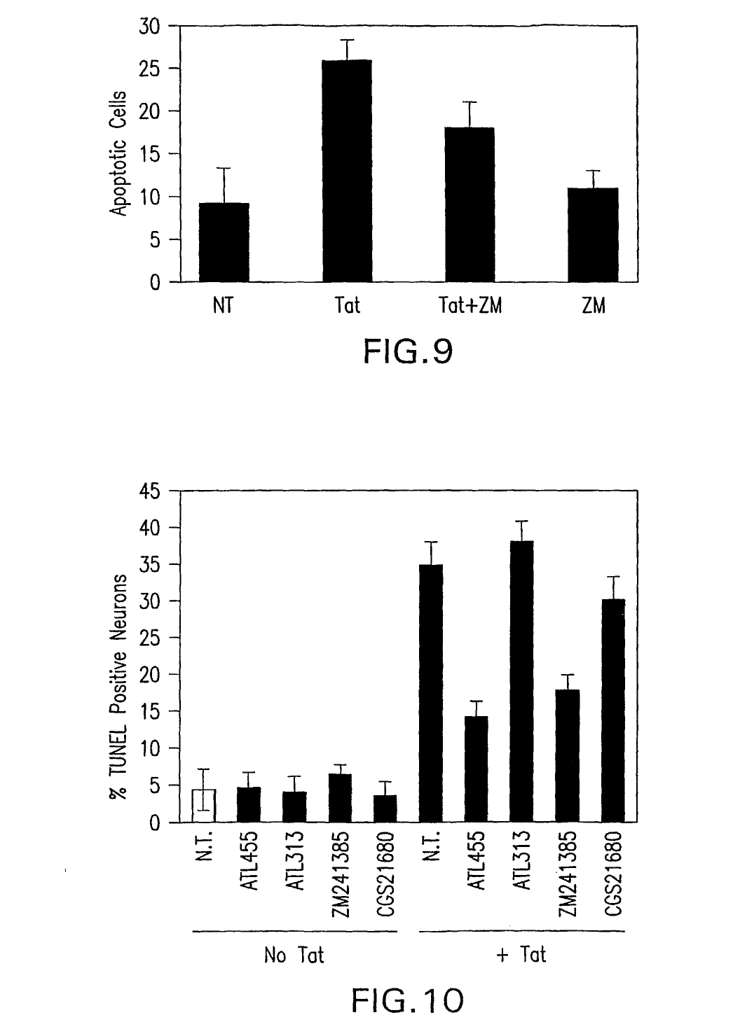

Primary rat cerebellar granule neurons (CGNs) were exposed to a neurotoxic concentration of HIV-1 Tat, in the presence or absence of an A_{2A}R antagonist (ZM241385). The A_{2A}R antagonist was able to protect neurons against the otherwise lethal effects of exposure to HIV-1 Tat (FIG. 9). These results demonstrate that A_{2A}R antagonists can have therapeutic potential in the context of HAD. It is important to note that CGNs express adenosine A_{2A} receptors [Vacas, J., et al. 2003. Brain Res 992:272-280].

Primary cultures of rat cerebellar granule neurons (CGNs) were exposed to HIV-Tat (500mM) alone or together with increasing doses of the A_{2A}R antagonist ATL-455 or A_{2A} agonists ATL313 or CGS21680. Cells are treated with the test compounds for defined time intervals of up to 48 hours (1, 4, 8, 24, and 48 hours). The A_{2A}R antagonist ATL455 protected neurons against Tat-induced apoptosis (FIG. 10). Further, exposure of neurons to an A_{2A}R agonists (ATL313 and CGS21680) did not result in an increase in cell death in the presence of Tat (FIG. 10).

RNAi can also be used to suppress expression of A_{2A}R and mimic the effects of A_{2A}R antagonist treatment. A_{2A}R expression is inhibited using lentivirus vectors that encode A_{2A}R-specific shRNA. CGN cultures are transduced with viral vectors encoding shRNA targeted to A_{2A}R, or scrambled shRNA sequences, or nothing (GFP only). For example, siRNA has been used to modify the levels of A_{2A}R and A_{2B}in cardiac fibroblasts (Chen Y, Epperson S, Makhsudova L, Ito B, Suarez J, Dillmann W, Villarreal F. Functional effects of enhancing or silencing adenosine A2b receptors in cardiac fibroblasts. Am J Physiol Heart Circ Physiol. 2004 December;287(6):H2478-86. Epub 2004 Jul. 29).

A_{2A}R-specific antibodies (Alpha Diagnostics International) are used to perform both immunoblot and immunofluorescence assays to examine knockdown of A_{2A}R. A knockdown of 80% or greater is followed by the treatment of the transduced neurons with candidate HIV-1 neurotoxins (or mock-treated, for controls).

The signal transduction pathways triggered by activation of A_{2A} receptors are still not completely understood. A_{2A} receptors are generally accepted to couple to the Gs-adenylate cyclase (AC)-protein kinase A (PKA) pathway [Fredholm, B. B., et al. 2001. Pharmacol Rev 53:527-552], but can also couple to pathways involving G-proteins other than Gs (Go, Gal5/16, Gi/o) or to cAMP PKA independent signal transduction pathways. Thus, to elucidating the signalling pathway(s) triggered by A_{2A} receptor antagonists, neurons are treated as described above, and the activity of cellular kinases such as PKA, ERK, AKT, JNK, and GSK-3 are evaluated.

The activation of transcription factors, e.g., AP-1, CREB, and NF-kB, is evaluated, as these factors represent potential endpoint targets of the signaling pathways initiated by A_{2A}R). Transcription factor activity is assessed using assays of protein nuclear translocation, nuclear DNA binding activity and transient transcriptional reporter assays. Antagonism of A_{2A}R signaling is expected to have a substantial effect on the activation of these transcription factors.

### Example 6

### A_{2A}R Agonists Prevent HIV-1 Induced Monocyte Activation

Microglial and macrophage activation contributes to neuronal damage in HIV-1 infected individuals [Poluektova, L., et al. 2004. J Immunol 172:7610-7617], and is associated with increased expression of inducible nitric oxide synthase (iNOS) and production of NO.

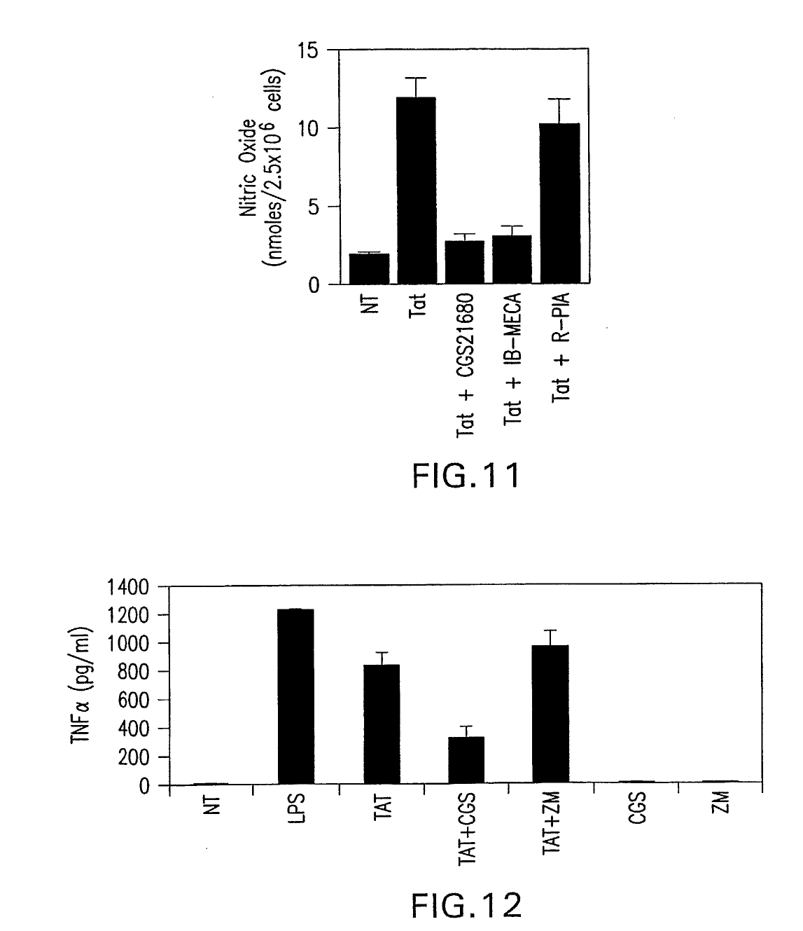

HIV-1 Tat produced a dose-dependent increase in iNOS expression in human primary monocytes, which was maximal at 100 nM. The effect of adenosine receptor activation on Tat-induced upregulation of iNOS expression and NO release was then determined. Tat-treatment resulted in a 4-fold increase in iNOS expression by primary monocytes and a similar elevation in NO release (FIG. 11). Co-treatment of cells with the A_{2A} adenosine receptor-selective agonist CGS21680 or the nonselective A₁ and A₃ adenosine receptor agonist IB-MECA resulted in a roughly 50% inhibition of Tat-induced iNOS expression and a complete abrogation of Tat's effect on NO secretion (FIG. 11). In contrast, incubation of cells with 1 μM R-PIA (an adenosine A₁ receptor agonist) had no effect on Tat-induced iNOS expression or NO release (FIG. 11), implicating the A_{2A} and A₃ receptor subtypes in this anti-inflammatory effect. ZM241385 (an A_{2A}R antagonist) or MRS 1220 (an A₃R antagonist) could prevent the anti-inflammatory effects of CGS21680 or IB-MECA, respectively.

The effect of an A_{2A}R agonist on TNF release by primary human monocytes was also determined. Monocytes were stimulated with HIV-1 Tat, in the presence or absence of the A_{2A}R agonist CGS21680 or the A_{2A}R antagonist ZM251385 and TNF release was then measured in cell culture supernatants using an ELISA assay. The results (FIG. 12) show that the A_{2A}R agonist abrogated the Tat-mediated increase in monocyte-derived TNF production.

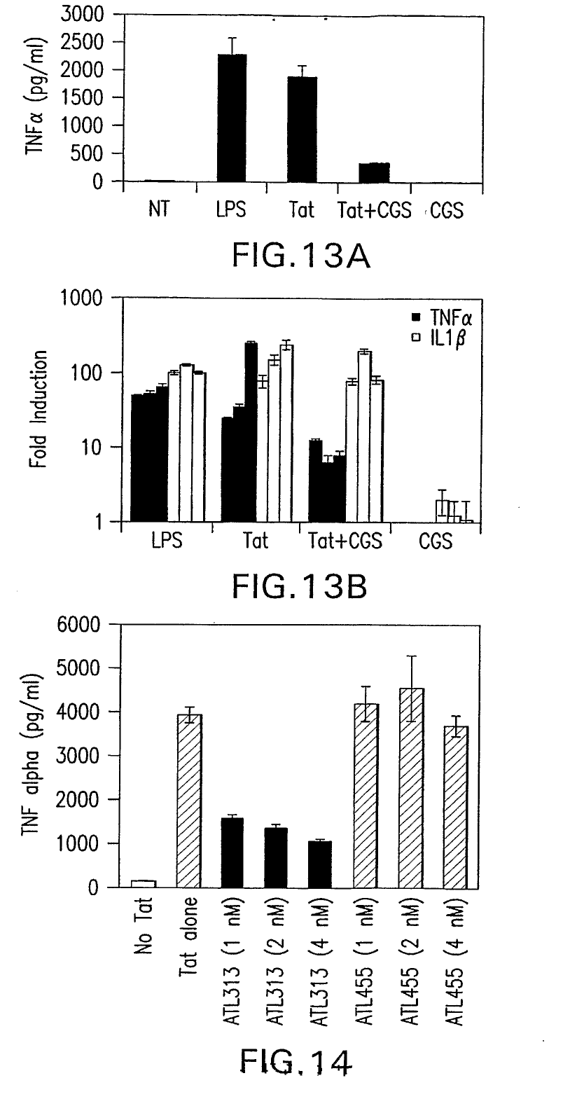

Quantitative real-time PCR analysis was used to measure TNF message levels in Tat-treated monocytes. Primary human monocytes were prepared using CD14⁺ immunomagnetic selection, and then exposed to LPS or to HIV-1 Tat for 4 hours, in the presence or absence of the A_{2A}R agonist CGS21680. Culture supernatants were tested for TNF levels by ELISA assay (FIG. 13A), and RNA was extracted from cell pellets for quantitative reverse-transcription PCR (qRTPCR) analysis (FIG. 13B).

Consistent with the results in FIG. 4, the A_{2A}R agonist CGS21680 strongly suppressed TNF release in Tat-exposed monocytes (FIG. 13B). Moreover, a strong concordance was observed between the TNF ELISA (FIG. 5A) and the qRTPCR analysis (FIG. 13B). Notably, the A_{2A}R agonist CGS21680 strongly suppressed TNF release (and TNF transcription) in Tat-exposed monocytes, but had little effect on IL1 mRNA levels in these cells (FIG. 13B).

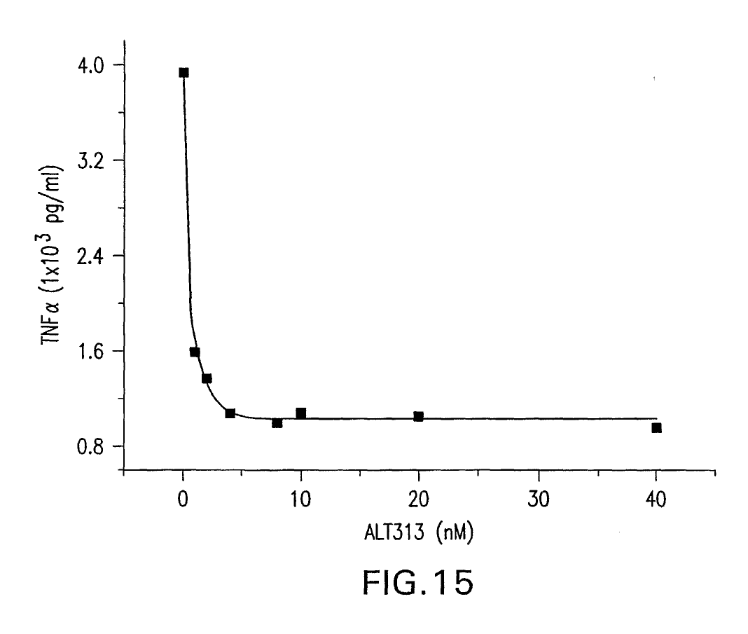

A second A_{2A}R agonist ATL313 demonstrated very similar results to those shown in FIG. 12 (see FIGS. 14 & 15). It was determined that exposure of monocytes to an A_{2A}R antagonist did not result in an increase in cellular activation in the presence of Tat (see data for ATL455; FIG. 6) and that the approximate IC50 for ATL313's inhibitory effect on Tat-stimulated TNF release in monocytes is <1 nM. This is consistent with the known receptor-binding properties of this compound (see Table 1). Collectively, the results shown in FIGS. 14 and 15 confirm and extend the findings in FIGS. 12 and 13, using a commercially relevant molecule (ATL313).

TABLE 3

| Receptor | A1 | A2A | A2B | A3 |
|---|---|---|---|---|
| ATL455 | 23 | 1.6 | 155 | 1000 |
| ATL313 | 52 | 0.6 | >1000 | 320 |

Data represent Ki (nM), versus recombinant versions of the indicated human adenosine receptor subtypes.

### REFERENCES

Adamson, D. C., et al., Neurovirulent simian immunodeficiency virus infection induces neuronal, endothelial, and glial apoptosis. Mol Med, 1996. 2(4): p. 417-28.

Adle-Biassette, H., et al., Neuronal apoptosis in HIV infection in adults. Neuropathol Appl Neurobiol, 1995. 21(3): p. 218-27.

Adle-Biassette, H., F. Chretien, L. Wingertsmann, C. Hery, T. Ereau, F. Scaravilli, M. Tardieu, and F. Gray. 1999. Neuronal apoptosis does not correlate with dementia in HIV infection but is related to microglial activation and axonal damage. Neuropathol Appl Neurobiol 25:123-133.

Albensi, B. C., and M. P. Mattson. 2000. Evidence for the involvement of TNF and NFkappaB in hippocampal synaptic plasticity. Synapse 35:151-159.

Anderson, E. R., H. E. Gendelman, and H. Xiong, Memantine protects hippocampal neuronal function in murine human immunodeficiency virus type 1 encephalitis. J Neurosci, 2004. 24(32): p. 7194-8.

Archibald, S. L., E. Masliah, C. Fennema-Notestine, T. D. Marcotte, R. J. Ellis, J. A. McCutchan, R. K. Heaton, I. Grant, M. Mallory, A. Miller, and T. L. Jernigan. 2004. Correlation of in vivo neuroimaging abnormalities with postmortem human immunodeficiency virus encephalitis and dendritic loss. Arch Neurol 61:369-376.

Arevalo, J. C., H. Yano, K. K. Teng, and M. V. Chao. 2004. A unique pathway for sustained neurotrophin signaling through an ankyrin-rich membrane-spanning protein. Embo J 23:2358-2368.

Barber, S. A., J. L. Uhrlaub, J. B. DeWitt, P. M. Tarwater, and M. C. Zink. 2004. Dysregulation of mitogen-activated protein kinase signaling pathways in simian immunodeficiency virus encephalitis. Am J Pathol 164:355-362.

Bazan, N. G., C. F. Zorumski, and G. D. Clark. 1993. The activation of phospholipase A2 and release of arachidonic acid and other lipid mediators at the synapse: the role of plateletactivating factor. J Lipid Mediat 6:421-427.

Beattie, E. C., et al., Control of synaptic strength by glial TNFalpha. Science, 2002. 295(5563): p. 2282-5.

Berger, J. R. and G. Arendt, HIV dementia: the role of the basal ganglia and dopaminergic systems. J Psychopharmacol, 2000. 14(3): p. 214-21.

Bissel, S. J., G. Wang, M. Ghosh, T. A. Reinhart, S. Capuano, 3rd, K. Stefano Cole, M. Murphey-Corb, M. Piatak Jr, Jr., J. D. Lifson, and C. A. Wiley. 2002. Macrophages relate presynaptic and postsynaptic damage in simian immunodeficiency virus encephalitis. Am J Pathol 160:927-941.

Bodner, A., A. C. Maroney, J. P. Finn, G. Ghadge, R. Roos, and R. J. Miller. 2002. Mixed lineage kinase 3 mediates gp120IIIB-induced neurotoxicity. J Neurochem 82:1424-1434.

Bouron, A. 2001. Modulation of spontaneous quantal release of neurotransmitters in the hippocampus. Prog Neurobiol 63:613-635.

Brewer, G. J., J. R. Torricelli, E. K. Evege, and P. J. Price. 1993. Optimized survival of hippocampal neurons in B27-supplemented Neurobasal, a new serum-free medium combination. J Neurosci Res 35:567-76.

Bruce-Keller, A. J., A. Chauhan, F. O. Dimayuga, J. Gee, J. N. Keller, and A. Nath. 2003. Synaptic transport of human immunodeficiency virus-Tat protein causes neurotoxicity and gliosis in rat brain. J Neurosci 23:8417-8422.

Carter, W. O., P. K. Narayanan, and J. P. Robinson, Intracellular hydrogen peroxide and superoxide anion detection in endothelial cells. J Leukoc Biol, 1994. 55(2): p. 253-8.

Cass, W. A., et al., HIV-1 protein Tat potentiation of methamphetamine-induced decreases in evoked overflow of dopamine in the striatum of the rat. Brain Res, 2003. 984(1-2): p. 133-42.

Chen, B. T., M. V. Avshalumov, and M. E. Rice, H(2)O(2) is a novel, endogenous modulator of synaptic dopamine release. J Neurophysiol, 2001. 85(6): p. 2468-76.

Cheng, J., et al., Neuronal excitatory properties of human immunodeficiency virus type 1 Tat protein. Neuroscience, 1998. 82(1): p. 97-106.

Clark, G. D., et al., Enhancement of hippocampal excitatory synaptic transmission by platelet-activating factor. Neuron, 1992. 9(6): p. 1211-6.

Colonno, R. J., et al., Activities of atazanavir (BMS-232632) against a large panel of human immunodeficiency virus type 1 clinical isolates resistant to one or more approved protease inhibitors. Antimicrob Agents Chemother, 2003. 47(4): p. 1324-33.

Dall'Igna, O. P., L. O. Porciuncula, D. O. Souza, R. A. Cunha, D. R. Lara, and O. P. Dall'Igna. 2003. Neuroprotection by caffeine and adenosine A_{2A} receptor blockade of betaamyloid neurotoxicity. Br J Pharmacol 138:1207-1209.

Day, Y. J., M. A. Marshall, L. Huang, M. J. McDuffie, M. D. Okusa, and J. Linden. 2004. Protection from ischemic liver injury by activation of A_{2A} adenosine receptors during reperfusion: inhibition of chemokine induction. Am J Physiol Gastrointest Liver Physiol 286:G285-293.

Diogenes, M. J., C. C. Fernandes, A. M. Sebastiao, and J. A. Ribeiro. 2004. Activation of adenosine A_{2A} receptor facilitates brain-derived neurotrophic factor modulation of synaptic transmission in hippocampal slices. J Neurosci 24:2905-2913.

Dou, H., et al., Neuroprotective activities of sodium valproate in a murine model of human immunodeficiency virus-1 encephalitis. J Neurosci, 2003. 23(27): p. 9162-70.

Elkabes, S., L. Peng, and I. B. Black. 1998. Lipopolysaccharide differentially regulates microglial trk receptor and neurotrophin expression. J Neurosci Res 54:117-122.

Everall, I. P. 2000. Neuronal damage—recent issues and implications for therapy. J Neurovirol 6 Suppl 1:S103-105.

Everall, I. P., R. K. Heaton, T. D. Marcotte, R. J. Ellis, J. A. McCutchan, J. H. Atkinson, I. Grant, M. Mallory, and E. Masliah. 1999. Cortical synaptic density is reduced in mild to moderate human immunodeficiency virus neurocognitive disorder. HNRC Group. HIV Neurobehavioral Research Center. Brain Pathol 9:209-217.

Everall, I. P., et al., Neuronal density in the superior frontal and temporal gyri does not correlate with the degree of human immunodeficiency virus-associated dementia. Acta Neuropathol (Berl), 1994. 88(6): p. 538-44.

Everall, I. P., P. J. Luthert, and P. L. Lantos, Neuronal loss in the frontal cortex in HIV infection. Lancet, 1991. 337(8750): p. 1119-21.

Fine, S. M., S. B. Maggirwar, P. R. Elliott, L. G. Epstein, H. A. Gelbard, and S. Dewhurst. 1999. Proteasome blockers inhibit TNF-alpha release by lipopolysaccharide stimulated macrophages and microglia: implications for HIV-1 dementia. J Neuroimmunol 95:55-64.

Fotheringham, J., M. Mayne, C. Holden, A. Nath, and J. D. Geiger. 2004. Adenosine receptors control HIV-1 Tat-induced inflammatory responses through protein phosphatase. Virology 327:186-195.

Fox, H. S., et al., Antiviral treatment normalizes neurophysiological but not movement abnormalities in simian immunodeficiency virus-infected monkeys. J Clin Invest, 2000. 106(1): p. 37-45.

Fredholm, B. B., and P. Svenningsson. 2003. Adenosine-dopamine interactions: development of a concept and some comments on therapeutic possibilities. Neurology 61:S5-9.

Fredholm, B. B., I. J. A P, K. A. Jacobson, K. N. Klotz, and J. Linden. 2001. International Union of Pharmacology. XXV. Nomenclature and classification of adenosine receptors. Pharmacol Rev 53:527-552.

Freudenthal, R., A. Romano, and A. Routtenberg. 2004. Transcription factor NF-kappaB activation after in vivo perforant path LTP in mouse hippocampus. Hippocampus 14:677-683.

Ganguly, A., T. F. Oo, M. Rzhetskaya, R. Pratt, O. Yarygina, T. Momoi, N. Kholodilov, and R. E. Burke. 2004. CEP11004, a novel inhibitor of the mixed lineage kinases, suppresses apoptotic death in dopamine neurons of the substantia nigra induced by 6-hydroxydopamine. J Neurochem 88:469-480.

Gelbard, H. A., et al., Identification of apoptotic neurons in post-mortem brain tissue with HIV-1 encephalitis and progressive encephalopathy. Neuropathol Appl Neurobiol, 1995. 21: p. 208-217.

Gelbard, H. A., et al., Platelet-activating factor: a candidate human immunodeficiency virus type 1-induced neurotoxin. J Virol, 1994. 68(7): p. 4628-35.

Gendelman, H. E., et al., Suppression of inflammatory neurotoxins by highly active antiretroviral therapy in human immunodeficiency virus-associated dementia. J Infect Dis, 1998. 178(4): p. 1000-7.

Giniatullin, A. R. and R. A. Giniatullin, Dual action of hydrogen peroxide on synaptic transmission at the frog neuromuscular junction. J Physiol, 2003. 552(Pt 1): p. 283-93.

Glass, J. D., H. Fedor, S. L. Wesselingh, and J. C. McArthur. 1995. Immunocytochemical quantitation of human immunodeficiency virus in the brain: correlations with dementia. Ann Neurol 38:755-762.

Gonzalez, R. G., et al., Early brain injury in the SIV-macaque model of AIDS. Aids, 2000. 14(18): p. 2841-9.

Gray, F., and C. Keohane. 2003. The neuropathology of HIV infection in the era of Highly Active AntiRetroviral Therapy (HAART). Brain Pathol 13:79-83.

Gray, F., et al., Neuronal apoptosis in human immunodeficiency virus infection. J Neurovirol, 2000. 6 Suppl 1: p. S38-43.

Green, K., M. D. Brand, and M. P. Murphy, Prevention of mitochondrial oxidative damage as a therapeutic strategy in diabetes. Diabetes, 2004. 53 Suppl 1: p. SI 10-8.

Gurwell, J. A., et al., Synergistic neurotoxicity of opioids and human immunodeficiency virus-1 Tat protein in striatal neurons in vitro. Neuroscience, 2001. 102(3): p. 555-63.

Haughey, N. J., C. P. Holden, A. Nath, and J. D. Geiger. 1999. Involvement of inositol 1,4,5-trisphosphate-regulated stores of intracellular calcium in calcium dysregulation and neuron cell death caused by HIV-1 protein tat. J Neurochem 73:1363-1374.

Hersh, B. P., P. R. Rajendran, and D. Battinelli, Parkinsonism as the presenting manifestation of HIV infection: improvement on HAART. Neurology, 2001. 56(2): p. 278-9.

Hidding, U., K. Mielke, V. Waetzig, S. Brecht, U. Hanisch, A. Behrens, E. Wagner, and T. Herdegen. 2002. The c-Jun N-terminal kinases in cerebral microglia: immunological functions in the brain. Biochem Pharmacol 64:781-788.

Hongisto, V., N. Smeds, S. Brecht, T. Herdegen, M. J. Courtney, and E. T. Coffey. 2003. Lithium blocks the c-Jun stress response and protects neurons via its action on glycogen synthase kinase 3. Mol Cell Biol 23:6027-6036.

James, H. J., et al., Expression of caspase-3 in brains from paediatric patients with HIV-1 encephalitis. Neuropathol Appl Neurobiol, 1999. 25(5): p. 380-6.

Jonas, E., Regulation of synaptic transmission by mitochondrial ion channels. J Bioenerg Biomembr, 2004. 36(4): p. 357-61.

Kadenbach, B., M. Huttemann, S. Arnold, I. Lee, and E. Bender. 2000. Mitochondrial energy metabolism is regulated via nuclear-coded subunits of cytochrome c oxidase. Free Radio Biol Med 29:211-221.

Kaku, K., Y. Inoue, and T. Kaneko, Extrapancreatic effects of sulfonylurea drugs. Diabetes Res Clin Pract, 1995. 28 Suppl: p. S105-8.

Kamsler, A. and M. Segal, Hydrogen peroxide as a diffusible signal molecule in synaptic plasticity. Mol Neurobiol, 2004. 29(2): p. 167-78.

Ketzler, S., et al., Loss of neurons in the frontal cortex in AIDS brains. Acta Neuropathol (Berl), 1990. 80(1): p. 92-4.

Kim, D. H., X. Zhao, C. H. Tu, P. Casaccia-Bonnefil, and M. V. Chao. 2004. Prevention of apoptotic but not necrotic cell death following neuronal injury by neurotrophins signaling through the tyrosine kinase receptor. J Neurosurg 100:79-87.

Kim, S. H., C. J. Smith, and L. J. Van Eldik. 2004. Importance of MAPK pathways for microglial pro-inflammatory cytokine IL-1 beta production. Neurobiol Aging 25:431-439.

Kloppenburg, M., B. M. Brinkman, H. H. de Rooij-Dijk, A. M. Miltenburg, M. R. Daha, F. C. Breedveld, B. A. Dijkmans, and C. Verweij. 1996. The tetracycline derivative minocycline differentially affects cytokine production by monocytes and T lymphocytes. Antimicrob Agents Chemother 40:934-940.

Kolson, D. L. and F. Gonzalez-Scarano, HIV and HIV dementia. J Clin Invest, 2000. 106(1): p. 11-3.

Koutsilieri, E., et al., Parkinsonism in HIV dementia. J Neural Transm, 2002. 109(5-6): p. 767-75.

Kovacs, A. D., S. Chakraborty-Sett, S. H. Ramirez, L. F. Sniderhan, A. L. Williamson, and S. B. Maggirwar. 2004. Mechanism of NF-kappaB inactivation induced by survival signal withdrawal in cerebellar granule neurons. Eur J Neurosci 20:345-352.

Krebs, F. C., H. Ross, J. McAllister, and B. Wigdahl. 2000. HIV-1-associated central nervous system dysfunction. Adv Pharmacol 49:315-385.

Krohn, A. J., T. Wahlbrink, and J. H. Prehn, Mitochondrial depolarization is not required for neuronal apoptosis. J Neurosci, 1999. 19(17): p. 7394-404.

Kruman, I I, A. Nath, and M. P. Mattson. 1998. HIV-1 protein Tat induces apoptosis of hippocampal neurons by a mechanism involving caspase activation, calcium overload, and oxidative stress. Exp Neurol 154:276-88.

Kusdra, L., D. McGuire, and L. Pulliam. 2002. Changes in monocyte/macrophage neurotoxicity in the era of HAART: implications for HIV-associated dementia. Aids 16:31-38.

Lambotte, O., K. Deiva, and M. Tardieu. 2003. HIV-1 persistence, viral reservoir, and the central nervous system in the HAART era. Brain Pathol 13:95-103.

Langford, T. D., S. L. Letendre, G. J. Larrea, and E. Masliah. 2003. Changing patterns in the neuropathogenesis of HIV during the HAART era. Brain Pathol 13:195-210.

Langford, T. D., et al., Changing patterns in the neuropathogenesis of HIV during the HAART era. Brain Pathol, 2003. 13(2): p. 195-210.

Langford, T. D., et al., Severe, demyelinating leukoencephalopathy in AIDS patients on antiretroviral therapy. Aids, 2002. 16(7): p. 1019-29.

Lee, F. S., R. Rajagopal, and M. V. Chao. 2002. Distinctive features of Trk neurotrophin receptor transactivation by G protein-coupled receptors. Cytokine Growth Factor Rev 13:11-17.

Lee, H. B., M. C. Zaccaro, M. Pattarawarapan, S. Roy, H. U. Saragovi, and K. Burgess. 2004. Syntheses and activities of new C10 beta-turn peptidomimetics. J Org Chem 69:701-713.

Liss, B. and J. Roeper, Molecular physiology of neuronal K-ATP channels (review). Mol Membr Biol, 2001. 18(2): p. 117-27.

Lu, Y., C. Liu, L. Zeng, Z. Lin, S. Dewhurst, S. Gartner, and V. Planelles. 2003. Efficient gene transfer into human monocyte-derived macrophages using defective lentiviral vectors. Cell Mol Biol (Noisy-le-grand) 49:1151-1156.

Ma, M. and A. Nath, Molecular determinants for cellular uptake of Tat protein of human immunodeficiency virus type 1 in brain cells. J Virol, 1997. 71(3): p. 2495-9.

Maggirwar, S. B., D. N. Dhanraj, S. M. Somani, and V. Ramkumar. 1994. Adenosine acts as an endogenous activator of the cellular antioxidant defense system. Biochem Biophys Res Commun 201:508-515.

Maggirwar, S. B., N. Tong, S. Ramirez, H. A. Gelbard, and S. Dewhurst. 1999. HIV-1 Tat-mediated activation of glycogen synthase kinase-3beta contributes to Tat-mediated neurotoxicity. J Neurochem 73:578-586.

Maggirwar, S. B., P. D. Sarmiere, S. Dewhurst, and R. S. Freeman. 1998. Nerve growth factor-dependent activation of NF-kappaB contributes to survival of sympathetic neurons. J Neurosci 18:10356-10365.

Maggirwar, S. B., S. Ramirez, N. Tong, H. A. Gelbard, and S. Dewhurst. 2000. Functional interplay between nuclear factor-kappaB and c-Jun integrated by coactivator p300 determines the survival of nerve growth factor-dependent PC12 cells. J Neurochem 74:527-539.

Magnuson, D. S., et al., Human immunodeficiency virus type 1 tat activates non-N-methyl-D-aspartate excitatory amino acid receptors and causes neurotoxicity. Ann Neurol, 1995. 37(3): p. 373-80.

Maragos, W. F., K. L. Young, J. T. Turchan, M. Guseva, J. R. Pauly, A. Nath, and W. A. Cass. 2002. Human immunodeficiency virus-1 Tat protein and methamphetamine interact synergistically to impair striatal dopaminergic function. J Neurochem 83:955-63.

Marathe, G. K., et al., Activation of vascular cells by PAF-like lipids in oxidized LDL. Vascul Pharmacol, 2002. 38(4): p. 193-200.

Masliah, E., et al., Changes in pathological findings at autopsy in AIDS cases for the last 15 years. Aids, 2000. 14(1): p. 69-74.

Masliah, E., R. K. Heaton, T. D. Marcotte, R. J. Ellis, C. A. Wiley, M. Mallory, C. L. Achim, J. A. McCutchan, J. A. Nelson, J. H. Atkinson, and I. Grant. 1997. Dendritic injury is a pathological substrate for human immunodeficiency virus-related cognitive disorders. HNRC Group. The HIV Neurobehavioral Research Center. Ann Neurol 42:963-972.

Matarrese, P., et al., Mitochondrial membrane hyperpolarization hijacks activated T lymphocytes toward the apoptotic-prone phenotype: homeostatic mechanisms of HIV protease inhibitors. J Immunol, 2003. 170(12): p. 6006-15.

Mattos, J. P., et al., Movement disorders in 28 HIV-infected patients. Arq Neuropsiquiatr, 2002. 60(3-A): p. 525-30.

Mattson, M. P. and D. Liu, Energetics and oxidative stress in synaptic plasticity and neurodegenerative disorders. Neuromolecular Med, 2002. 2(2): p. 215-31.

Mattson, M. P. and D. Liu, Mitochondrial potassium channels and uncoupling proteins in synaptic plasticity and neuronal cell death. Biochem Biophys Res Commun, 2003. 304(3): p. 539-49.

Mattson, M. P., J. N. Keller, and J. G. Begley, Evidence for synaptic apoptosis. Exp Neurol, 1998.153(1): p. 35-48.

Meffert, M. K., J. M. Chang, B. J. Wiltgen, M. S. Fanselow, and D. Baltimore. 2003. NFkappa B functions in synaptic signaling and behavior. Nat Neurosci 6:1072-1078.

Meier, J. J., et al., Is impairment of ischaemic preconditioning by sulfonylurea drugs clinically important? Heart, 2004. 90(1): p. 9-12.

Mota, M., M. Reeder, J. Chemoff, and C. E. Bazenet. 2001. Evidence for a role of mixed lineage kinases in neuronal apoptosis. J Neurosci 21:4949-4957.

Murphy, M. P., and M. D. Brand. 1988. The stoichiometry of charge translocation by cytochrome oxidase and the cytochrome bc1 complex of mitochondria at high membrane potential. Eur J Biochem 173:645-651.

Nath, A., and J. Berger. 2004. HIV Dementia. Curr Treat Options Neurol 6:139-151.

Nath, A., K. Psooy, C. Martin, B. Knudsen, D. S. Magnuson, N. Haughey, and J. D. Geiger. 1996. Identification of a human immunodeficiency virus type 1 Tat epitope that is neuroexcitatory and neurotoxic. J Virol 70:1475-80.

New, D. R., M. Ma, L. G. Epstein, A. Nath, and H. A. Gelbard. 1997. Human immunodeficiency virus type 1 Tat protein induces death by apoptosis in primary human neuron cultures. J Neurovirol 3:168-73.

New, D. R., et al., HIV-1 Tat induces neuronal death via tumor necrosis factor-alpha and activation of non-N-methyl-D-aspartate receptors by a NFkappaB-independent mechanism. J Biol Chem, 1998. 273(28): p. 17852-8.

Nicholls, D. G., et al., Interactions between mitochondrial bioenergetics and cytoplasmic calcium in cultured cerebellar granule cells. Cell Calcium, 2003. 34(4-5): p. 407-24.

Nicholls, D. G., Mitochondrial function and dysfunction in the cell: its relevance to aging and aging related disease. Int J Biochem Cell Biol, 2002. 34(11): p. 1372-81.

Nuovo, G. J., et al., In situ detection of polymerase chain reaction-amplified HIV-1 nucleic acids and tumor necrosis factor-alpha RNA in the central nervous system. Am J Pathol, 1994. 144(4): p. 659-66.

Ochi, M., S. Shiozaki, and H. Kase, Adenosine A(2A) receptor-mediated modulation of GABA and glutamate release in the output regions of the basal ganglia in a rodent model of Parkinson's disease. Neuroscience, 2004. 127(1): p. 223-31.

O'Leary, P. D., and R. A. Hughes. 2003. Design of potent peptide mimetics of brain derived neurotrophic factor. J Biol Chem 278:25738-25744.

Papa, S., F. Zazzeroni, C. Bubici, S. Jayawardena, K. Alvarez, S. Matsuda, D. U. Nguyen, C. G. Pham, A. H. Nelsbach, T. Melis, E. De Smaele, W. J. Tang, L. D'Adamio, and G. Franzoso. 2004. Gadd45 beta mediates the NF-kappa B suppression of JNK signalling by targeting MKK7/JNKK2. Nat Cell Biol 6:146-153.

Papa, S., F. Zazzeroni, C. G. Pham, C. Bubici, and G. Franzoso. 2004. Linking JNK signaling to NF-{kappa}B: a key to survival. J Cell Sci 117:5197-5208.

Patrizio, M., M. Colucci, and G. Levi. 2001. Human immunodeficiency virus type I Tat protein decreases cyclic AMP synthesis in rat microglia cultures. J Neurochem 77:399-407.

Perry, S. W., J. A. Hamilton, L. W. Tjoelker, G. Dbaibo, K. A. Dzenko, L. G. Epstein, Y. Hannun, J. S. Whittaker, S. Dewhurst, and H. A. Gelbard. 1998. Platelet-activating factor receptor activation. An initiator step in HIV- 1 neuropathogenesis. J Biol Chem 273:17660-17664.

Perry, S. W., et al., Antioxidants are required during the early critical period, but not later, for neuronal survival. J Neurosci Res, 2004. 78(4): p. 485-92.

Perry, S. W., L. G. Epstein, and H. A. Gelbard, Simultaneous in situ detection of apoptosis and necrosis in monolayer cultures by TUNEL and trypan blue staining. Biotechniques, 1997.22(6): p. 1102-6.

Petito, C. K. and B. Roberts, Evidence of apoptotic cell death in HIV encephalitis. Am J Pathol, 1995.146(5): p. 1121-30.

Pi, R., et al., Minocycline prevents glutamate-induced apoptosis of cerebellar granule neurons by differential regulation of p38 and Akt pathways. J Neurochem, 2004. online early.

Piacentini, M., et al., Transglutaminase overexpression sensitizes neuronal cell lines to apoptosis by increasing mitochondrial membrane potential and cellular oxidative stress. J Neurochem, 2002. 81(5): p. 1061-72.

Poluektova, L., S. Gorantla, J. Faraci, K. Birusingh, H. Dou, and H. E. Gendelman. 2004. Neuroregulatory events follow adaptive immune-mediated elimination of HIV-1-infected macrophages: studies in a murine model of viral encephalitis. J Immunol 172:7610-7617.

Poppe, M., et al., Dissipation of potassium and proton gradients inhibits mitochondrial hyperpolarization and cytochrome c release during neural apoptosis. J Neurosci, 2001. 21(13): p. 4551-63.

Prange, O. and T. H. Murphy, Correlation of miniature synaptic activity and evoked release probability in cultures of cortical neurons. J Neurosci, 1999. 19(15): p. 6427-38.

Pyle, J. L., et al., Visualization of synaptic activity in hippocampal slices with FM1-43 enabled by fluorescence quenching. Neuron, 1999. 24(4): p. 803-8.

Rajagopal, R., Z. Y. Chen, F. S. Lee, and M. V. Chao. 2004. Transactivation of Trk neurotrophin receptors by g-protein-coupled receptor ligands occurs on intracellular membranes. J Neurosci 24:6650-6658.

Ramirez, S. H., J. F. Sanchez, C. A. Dimitri, H. A. Gelbard, S. Dewhurst, and S. B. Maggirwar. 2001. Neurotrophins prevent HIV Tat-induced neuronal apoptosis via a nuclear factor-kappaB (NF-kappaB)-dependent mechanism. J Neurochem 78:874-889.

Ramirez, S. H., S. Fan, C. A. Maguire, S. Perry, K. Hardiek, V. Ramkumar, H. A. Gelbard, S. Dewhurst, and S. B. Maggirwar. 2004. Activation of adenosine A_{2A} receptor protects sympathetic neurons against nerve growth factor withdrawal. J Neurosci Res 77:258-269.

Rintoul, G. L., et al., Glutamate decreases mitochondrial size and movement in primary forebrain neurons. J Neurosci, 2003. 23(21): p. 7881-8.

Riveline, J. P., et al., Sulfonylureas and cardiovascular effects: from experimental data to clinical use. Available data in humans and clinical applications. Diabetes Metab, 2003. 29(3): p. 207-22.

Rottenberg, H. and S. Wu, Quantitative assay by flow cytometry of the mitochondrial membrane potential in intact cells. Biochim Biophys Acta, 1998. 1404(3): p. 393-404.

Roux, P. P., G. Dorval, M. Boudreau, A. Angers-Loustau, S. J. Morris, J. Makkerh, and P. A. Barker. 2002. K252a and CEP1347 are neuroprotective compounds that inhibit mixed-lineage kinase-3 and induce activation of Akt and ERK. J Biol Chem 277:49473-49480.

Ryan, L. A., et al., Macrophages, chemokines and neuronal injury in HIV-1-associated dementia. Cell Mol Biol (Noisy-le-grand), 2002. 48(2): p. 137-50.

Ryu, J. K., et al., ATP-induced in vivo neurotoxicity in the rat striatum via P2 receptors. Neuroreport, 2002. 13(13): p. 1611-5.

Sacktor, N. 2002. The epidemiology of human immunodeficiency virus-associated neurological disease in the era of highly active antiretroviral therapy. J Neurovirol 8 Suppl 2:115-121.

Sanchez, J. F., L. F. Sniderhan, A. L. Williamson, S. Fan, S. Chakraborty-Sett, and S. B. Maggirwar. 2003. Glycogen synthase kinase 3beta-mediated apoptosis of primary cortical astrocytes involves inhibition of nuclear factor kappaB signaling. Mol Cell Biol 23:4649-4662.

Schwanstecher, C. and D. Bassen, KATP-channel on the somata of spiny neurones in rat caudate nucleus: regulation by drugs and nucleotides. Br J Pharmacol, 1997. 121(2): p. 193-8.

Si, Q., M. Cosenza, M. 0. Kim, M. L. Zhao, M. Brownlee, H. Gold stein, and S. Lee. 2004. A novel action of minocycline: inhibition of human immunodeficiency virus type 1 infection in microglia. J Neurovirol 10:284-292.

Smith, P. A., P. Proks, and A. Moorhouse, Direct effects of tolbutamide on mitochondrial function, intracellular Ca2+and exocytosis in pancreatic beta-cells. Pflugers Arch, 1999. 437(4): p. 577-88.

Smith, R. A., et al., Delivery of bioactive molecules to mitochondria in vivo. Proc Natl Acad Sci U S A, 2003.100(9): p. 5407-12.

Song, L., A. Nath, J. D. Geiger, A. Moore, and S. Hochman. 2003. Human immunodeficiency virus type 1 Tat protein directly activates neuronal N-methyl-D-aspartate receptors at an allosteric zinc-sensitive site. J Neurovirol 9:399-403.

Soontomniyomkij, V., G. Wang, C. A. Pittman, R. L. Hamilton, C. A. Wiley, and C. L. Achim. 1999. Absence of brain-derived neurotrophic factor and trkB receptor intnunoreactivity in glia of Alzheimer's disease. Acta Neuropathol (Berl) 98:345-348.

Spencer, J. P. and K. P. Murphy, Bi-directional changes in synaptic plasticity induced at corticostriatal synapses in vitro. Exp Brain Res, 2000. 135(4): p. 497-503.

Sullivan, G. W. 2003. Adenosine A_{2A} receptor agonists as anti-inflammatory agents. Curr Opin Investig Drugs 4:1313-1319.

Takahashi, K., et al., Localization of HIV-1 in human brain using polymerase chain reaction/in situ hybridization and immunocytochemistry. Ann Neurol, 1996. 39(6): p. 705-11.

Tang, G., Y. Minemoto, B. Dibling, N. H. Purcell, Z. Li, M. Karin, and A. Lin. 2001. Inhibition of JNK activation through NF-kappaB target genes. Nature 414:313-317.

Tanganelli, S., et al., Striatal plasticity at the network level. Focus on adenosine A_{2A} and D2 interactions in models of Parkinson's Disease. Parkinsonism Relat Disord, 2004. 10(5): p. 273-80.

Teng, Y. D., et al., Minocycline inhibits contusion-triggered mitochondrial cytochrome c release and mitigates functional deficits after spinal cord injury. Proc Natl Acad Sci U S A, 2004. 101(9): p. 3071-6.

Tenhula, W. N., et al., Morphometric comparisons of optic nerve axon loss in acquired immunodeficiency syndrome. Am J Ophthalmol, 1992. 113(1): p. 14-20.

Thiele, A., R. Kronstein, A. Wetzel, A. Gerth, K. Nieber, and S. Hauschildt. 2004. Regulation of adenosine receptor subtypes during cultivation of human monocytes: role of receptors in preventing lipopolysaccharide-triggered respiratory burst. Infect Immun 72:1349-1357.

Tong, N., J. F. Sanchez, S. B. Maggirwar, S. H. Ramirez, H. Guo, S. Dewhurst, and H. A. Gelbard. 2001. Activation of glycogen synthase kinase 3 beta (GSK-3beta) by platelet activating factor mediates migration and cell death in cerebellar granule neurons. Eur J Neurosci 13:1913-22.

Tse, W., et al., Movement disorders and AIDS: a review. Parkinsonism Relat Disord, 2004. 10(6): p. 323-34.

Uhl, E., et al., Influence of platelet-activating factor on cerebral microcirculation in rats: part 2. Local application. Stroke, 1999. 30(4): p. 880-6.

Vacas, J., M. Fernandez, M. Ros, and P. Blanco. 2003. Adenosine modulation of [Ca²⁺] in cerebellar granular cells: multiple adenosine receptors involved. Brain Res 992:272-280.

Valcour, V. G., C. M. Shikuma, M. R. Watters, and N. C. Sacktor. 2004. Cognitive impairment in older HIV-1-seropositive individuals: prevalence and potential mechanisms. Aids 18 Suppl 1:S79-86.

Wakade, T. D., et al. Adenosine-induced apoptosis in chick embryonic sympathetic neurons: a new physiological role for adenosine. J Physiol, 1995. 488 (Pt 1): p. 123-38.

Wang, W., L. Shi, Y. Xie, C. Ma, W. Li, X. Su, S. Huang, R. Chen, Z. Zhu, Z. Mao, Y. Han, and M. Li. 2004. SP600125, a new JNK inhibitor, protects dopaminergic neurons in the MPTP model of Parkinson's disease. Neurosci Res 48:195-202.

Wang, X., et al., P2X7 receptor inhibition improves recovery after spinal cord injury. Nat Med, 2004. 10(8): p. 821-7.

Wang, X., M. Messerle, R. Sapinoro, K. Santos, P. K. Hocknell, X. Jin, and S. Dewhurst. 2003. Murine cytomegalovirus abortively infects human dendritic cells, leading to expression and presentation of virally vectored genes. J Virol 77:7182-92.

Ward, M. W., et al., Mitochondrial membrane potential and glutamate excitotoxicity in cultured cerebellar granule cells. J Neurosci, 2000. 20(19): p. 7208-19.

Wardas, J. 2002. Neuroprotective role of adenosine in the CNS. Pol J Pharmacol 54:313-326.

Wiley, C. A., et al., Cellular localization of human immunodeficiency virus infection within the brains of acquired immune deficiency syndrome patients. Proc Natl Acad Sci U S A, 1986. 83(18): p. 7089-93.

Xie, Y., Y. Liu, C. Ma, Z. Yuan, W. Wang, Z. Zhu, G. Gao, X. Liu, H. Yuan, R. Chen, S. Huang, X. Wang, X. Zhu, Z. Mao, and M. Li. 2004. Indirubin-3′-oxime inhibits c-Jun NH2-terminal kinase: anti-apoptotic effect in cerebellar granule neurons. Neurosci Lett 367:355-359.

Xiong, H., et al., Activation of NR1a/NR2B receptors by monocyte-derived macrophage secretory products: implications for human immunodeficiency virus type one-associated dementia. Neurosci Lett, 2003. 341(3): p. 246-50.

Yang, D. D., C. Y. Kuan, A. J. Whitmarsh, M. Rincon, T. S. Zheng, R. J. Davis, P. Rakic, and R. A. Flavell. 1997. Absence of excitotoxicity-induced apoptosis in the hippocampus of mice lacking the Jnk3 gene. Nature 389:865-870.

Yoshida et. al., beta 3-Adrenergic agonist induces a functionally active uncoupling protein in fat and slow-twitch muscle fibers. Am J Physiol, 1998. 274(3 Pt 1): p. E469-75.

Yrjanheikki, J., R. Keinanen, M. Pellikka, T. Hokfelt, and J. Koistinaho. 1998. Tetracyclines inhibit microglial activation and are neuroprotective in global brain ischemia. Proc Natl Acad Sci U S A 95:15769-15774.

Yu, T. P., et al., Gamma-aminobutyric acid type A receptors modulate cAMP-mediated long-term potentiation and long-term depression at monosynaptic CA3-CA1 synapses. Proc Natl Acad Sci U S A, 2001. 98(9): p. 5264-9.

Zauli, G., et al., HIV-1 Tat-mediated inhibition of the tyrosine hydroxylase gene expression in dopaminergic neuronal cells. J Biol Chem, 2000. 275(6): p. 4159-65.

Zhang, J., C. Geula, C. Lu, H. Koziel, L. M. Hatcher, and F. J. Roisen. 2003. Neurotrophins regulate proliferation and survival of two microglial cell lines in vitro. Exp Neurol 183:469-481.

Zheng, J., M. R. Thylin, R. L. Cotter, A. L. Lopez, A. Ghorpade, Y. Persidsky, H. Xiong, G. B. Leisman, M. H. Che, and H. E. Gendelman. 2001. HIV-1 infected and immune competent mononuclear phagocytes induce quantitative alterations in neuronal dendritic arbor: relevance for HIV-l-associated dementia. Neurotox Res 3:443-59.

We claim:

1. A method of protecting a brain cell from synaptic dysfunction induced by an HIV neurotoxin, comprising contacting the cell with a therapeutically effective dose of an inhibitor of mitochondrial hyperpolarization.

2. The method of claim 1, wherein synaptic dysfunction results in neuronal cell death.

3. The method of claim 1, wherein the inhibitor is a KATP antagonist.

4. The method of claim 3, wherein the KATP antagonist is selected from the group consisting of Tolbutamide, hydroxydecanoic acid (5-HD), glibenclamide (glyburide), and meglitinide analog (e.g. Repaglinide, A-4166).

5. The method of claim 1, wherein the inhibitor is an inhibitor of electron transport.

6. The method of claim 5, wherein the inhibitor is selected from the group consisting of diphenyleneiodonium (DPI), rotenone, antimycin, myxothiazole, succinate-q reductase (TTFA), and potassium cyanide (KCN).

7. The method of claim 1, wherein the inhibitor is a protonophore.

8. The method of claim 7, wherein the protonophore is selected from the group consisting of Trifluorocarbonylcyanide Phenylhydrazone (FCCP), dinitrophenol (DNP), m-chlorophenylhydrazone (CCCP), and pentachlorophenol (PCP).

9. The method of claim 1, further comprising contacting the cell with an antioxidant.

10. The method of claim 9, wherein the antioxidant is selected from the group consisting of tauroursodeoxycholic acid (TUDCA), N-acetylcysteine (NAC), Mito-Coenzyme Q10 (Mito-CoQ), Mito-VitaminE (Mito-E), Coenzyme Q10, and ibedenone.

11. The method of claim 1, further comprising contacting the cell with an antiretroviral compound.

12. The method of claim 11, wherein the antiretroviral compound comprises one or more molecules selected from the group consisting of protease inhibitors [PI], nucleoside reverse transcriptase inhibitors [NRTI], and non-nucleoside reverse transcriptase inhibitors [NNRTI].

13. The method of claim 12, wherein the PI is selected from the group consisting of Indinavir, Amprenavir, Nelfinavir, Saquinavir, Fosamprenavir, Lopinavir, Ritonavir, and Atazanavir.

14. The method of claim 12, wherein the NRTI is selected from the group consisting of Abacavir, Stavudine, Didanosine, Lamivudine, Zidovudine, Zalcitabine, Tenofovir, and Emtricitabine.

15. The method of claim 12, wherein the NNRTI is selected from the group consisting of Efavirenz, Nevirapine, and Delavirdine.

16. A method of treating or preventing neurological disease in a subject in need of such treatment or prevention, comprising administering to the subject a therapeutically effective dose of an inhibitor of mitochondrial hyperpolarization.

17. The method of claim 16, wherein the neurological disease is HIV-1 associated dementia (HAD).

18. The method of claim 16, wherein the neurological disease is minor cognitive minor motor disease (MCMD).

19. The method of claim 16, wherein the mitochondrial hyperpolarization is induced by HIV-1 Tat.

20. A method of identifying a compound that can promote neural cell protection, the method comprising:
a. contacting a neural cell with a candidate neural protecting compound,
b. contacting the neural cell with an agent that induces mitochondrial hyperpolarization, and
c. evaluating the ability of the compound to prevent or inhibit mitochondrial hyperpolarization in the cell.

21. The method of claim 20, wherein a decrease in vesicle recycling, mitochondrial membrane potential, ATP/ADP ratios, NADH/NAD+ ratios, reactive oxygen species (ROS), dendritic beading, and cell death indicates inhibition of hyperpolarization in the cell.

22. The method of claim 20, wherein the neural cell is from a primary cell culture of cerebellar granule neurons (CGNs), cortical neurons (CN), hippocampal neurons or fetal neurons.

23. The method of claim 20, wherein the candidate neural protecting molecule is a KATP antagonist, an inhibitor of electron transport, a protonophore, or an antioxidant.

24. The method of claim 20, wherein the agent is a neurotoxin.

25. The method of claim 24, wherein the neurotoxin is an HIV neurotoxin.

26. The method of claim 25, wherein the neurotoxin is HIV-Tat or carbamyl-platelet-activating factor (c-PAF).

27. The method of claim 20, wherein the candidate compound is produced.

28. A compound produced by the method of claim 20.

29. A composition comprising a molecule that inhibits mitochondrial hyperpolarization in a neural cell and an antiretroviral compound.

30. The composition of claim 29, wherein the antiretroviral compound comprises one or more molecules selected from the group consisting of protease inhibitors [PI], nucleoside reverse transcriptase inhibitors [NRTI], and non-nucleoside reverse transcriptase inhibitors [NNRTI].

31. The composition of claim 30, wherein the PI is selected from the group consisting of Indinavir, Amprenavir, Nelfinavir, Saquinavir, Fosamprenavir, Lopinavir, Ritonavir, and Atazanavir.

32. The composition of claim 30, wherein the NRTI is selected from the group consisting of Abacavir, Stavudine, Didanosine, Lamivudine, Zidovudine, Zalcitabine, Tenofovir, and Emtricitabine.

33. The composition of claim 30, wherein the NNRTI is selected from the group consisting of Efavirenz, Nevirapine, and Delavirdine.
